(19) United States
(12) Patent Application Publication
(10) Pub. No.: US 2025/0367289 A1
(43) Pub. Date: Dec. 4, 2025

(54) DOSAGE REGIMEN FOR A COMBINATION THERAPY CONSISTING OFTCR-ENGINEERED T-CELLS IN COMBINATION WITH A PD-1 AXIS BINDING ANTAGONIST

(71) Applicant: Adaptimmune Limited, Abingdon, Oxfordshire (GB)

(72) Inventors: Jose SARO, Abingdon, Oxfordshire (GB); Steve DAWE, Abingdon, Oxfordshire (GB); Alejandro GARCIA, Abingdon, Oxfordshire (GB)

(21) Appl. No.: 18/874,958

(22) PCT Filed: Jun. 15, 2023
(86) PCT No.: PCT/GB2023/051565

§ 371 Date: Dec. 13, 2024

(30) Foreign Application Priority Data
Jun. 16, 2022 (GB) 2208849.6
Aug. 24, 2022 (GB) 2212323.6
Sep. 7, 2022 (GB) 2213032.2

(51) Int. Cl. A61K 40/11; A61K 39/00; A61K 39/395; A61K 40/32; A61K 40/42; A61P 35/00

(52) U.S. Cl. CPC A61K 40/11; A61K 39/39558; A61K 40/32; A61K 40/4268; A61P 35/00; A61K 2039/505; A61K 2039/545

(57) ABSTRACT

The disclosure relates to a method of treating cancer, and to a population of modified T cells for use in a method of treating cancer.

### FIELD OF THE DISCLOSURE

The disclosure relates to a method of treating cancer, to a population of modified T cells for use in a method of treating cancer, and to a PD-1 axis binding antagonist for use in a method of treating cancer.

### BACKGROUND

Immunotherapeutics are an important component of the anti-cancer tool kit. Immune effectors, such as antitumour monoclonal antibodies, T cells expressing a chimeric antigen receptor (CAR T cells), and TCR-engineered T cells may be adoptively transferred to an individual to promote an anti-cancer immune response and thereby treat disease.

The therapeutic capacity of immunotherapeutics may, however, be limited by the ability of cancer cells to modulate immune responses. It is important to maintain an activated and sustained T cell response in order to effectively eliminate a tumour. However, solid tumours possess an immunosuppressive tumour microenvironment that is promoted by cancer cells themselves and by infiltration of suppressive immune cells such as myeloid-derived suppressor cells (MDSCs) and regulatory T cells (Tregs). The immunosuppressive microenvironment may counteract anti-tumour T cell responses and/or promote T cell exhaustion, making it challenging to maintain an activated T cell response and eliminate the tumour.

There is therefore a need to develop dosage regimes for immunotherapeutic T cells that promote T cell survival and function, to overcome the immunosuppressive tumour microenvironment.

### SUMMARY OF THE DISCLOSURE

The present inventors have identified that improved anti-tumour immune responses may be obtained by administering tumour-specific immunotherapeutic T cells in combination with a PD-1 axis binding antagonist. In particular, the inventors propose that administration of a PD-1 axis binding antagonist may sustain the activity of endogenous T cells present in the immunosuppressive tumour microenvironment. The inventors further propose that such administration helps adoptively-transferred tumour-specific T cells to maintain their function including their ability to transition to memory T cells. In this way, the therapeutic effect of adoptively-transferred tumour-specific T cells may be enhanced and/or prolonged. The present inventors have further identified an optimal dosage regime for such combination therapy.

Accordingly, the disclosure provides a method of treating cancer in an individual, comprising (a) administering to the individual a population of modified T cells comprising a heterologous CD8 co-receptor and a heterologous TCR capable of binding to MAGE-A4; and (b) sustaining the function of (i) the population of modified T cells and/or (ii) endogenous T cells in the individual by administering an initial dose of a PD-1 axis binding antagonist to the individual about three weeks to about five weeks after step (a). The disclosure also provides:
a population of modified T cells for use in a method of treating cancer in an individual, wherein the modified T cells comprise a heterologous CD8 co-receptor and a heterologous TCR capable of binding to MAGE-A4, and the method comprises: (a) administering the population to the individual; and (b) sustaining the function of (i) the population of modified T cells and/or (ii) endogenous T cells in the individual by administering an initial dose of a PD-1 axis binding antagonist to the individual about three weeks to about five weeks after step (a);
a PD-1 axis binding antagonist for use in a method of treating cancer in an individual, wherein the method comprises: (a) administering to the individual a population of modified T cells comprising a heterologous CD8 co-receptor and a heterologous TCR capable of binding to MAGE-A4; and (b) sustaining the function of (i) the population of modified T cells and/or (ii) endogenous T cells in the individual by administering an initial dose of the PD-1 axis binding antagonist to the individual about three weeks to about five weeks after step (a).

### BRIEF DESCRIPTION OF THE FIGURES

FIG. 1: Scheme for evaluating monotherapy with modified T cells comprising a heterologous CD8 co-receptor and a heterologous TCR capable of binding to MAGE-A4.

FIG. 2: Scheme for evaluating combination therapy with a PD-1 axis antagonist and modified T cells comprising a heterologous CD8 co-receptor and a heterologous TCR capable of binding to MAGE-A4.

FIG. 3: Spider plots showing persistence of ADP-A2M4CD8 T cells. The X-axis shows duration in weeks (wherein 0 represents the day of T cell infusion) and the Y-axis represents the presence of ADP-A2M4CD8 T cells, in terms of vector copies per microgram DNA. Dotted line shows day 28. (A) All studied tumour types. (B) All tumour types excluding esophageal, esophagogastric junction, gastric and ovarian tumour type. (C) Head and neck tumours. (D) Esophagogastric junction and esophageal tumours. (E) Ovarian tumours.

FIG. 4: Comparative prior art data showing persistence of CD19 CAR T cells. Peak expansion of CAR T cells occurred within the first 7-14 days after infusion. Figure is from Cao et al. (2019), Anti-CD19 Chimeric Antigen Receptor T Cells in Combination With Nivolumab Are Safe and Effective Against Relapsed/Refractory B-Cell Non-hodgkin Lymphoma, Frontiers in Oncology, 9:767, doi: 10.3389/fonc.2019.00767.

FIG. 5: Spider plot showing clinical response to ADP-A2M4CD8 T cell administration. The X-axis shows duration in weeks (wherein 0 represents the day of T cell infusion) and the Y-axis represents percentage change in the sum of diameters of target lesions (compared to the baseline). Data are categorised by the BoR (Best Objective Response), as calculated based on the overall visit responses obtained up until RECIST progression is documented. In the absence of RECIST progression, BOR is determined using visit responses up until the last evaluable overall visit response. BOR represents the best response a patient has had during this time. BOR may be defined as: (1) Progressive Disease (PD): At least a 20% increase in the sum of the diameters of target lesions compared to the smallest sum of diameters previously recorded, plus an absolute increase of >=5 mm compared to the smallest sum previously recorded. Smallest sum of diameters (previous minimum) will be based on the sum of diameters at any previous visit including baseline (dotted red line). (2) Complete Response (CR): Disappearance of all target lesions since baseline. Any pathological lymph nodes selected as target lesions must have a reduction in short axis to <10 mm. (3) Partial Response (PR): At least a 30% decrease in the sum of the diameters of target lesions compared to the baseline sum of diameters (dotted green line). (4) Stable Disease (SD): Neither sufficient tumour shrinkage to qualify for PR nor sufficient increase to qualify for PD. (5) Not Evaluable (NE): Insufficient data is available to assign a target lesion response. (6) uPR (Unconfirmed PR) (i.e. a single overall visit response of PR).

FIG. 6: Intra-tumoral ADP-A2M4CD8 T cells are detected in 75% of evaluable biopsies obtained from patients administered with ADP-A2M4CD8 T cells.

(A) Total number of T cells (i.e., CD3+ cells) per mm², and number of ADP-A2M4CD8 T cells per mm², in biopsies from patients with various types of cancer. The data represents post-infusion data from 8 samples from 5 different indication types. Symbols represent the different tumour types. Lines between the symbols indicate matched patient data sets.

This figure shows the density of CD3+ (immune cells) in the post-infusion tissue only. The total number of T cells (left boxplot) is a combination of both modified (ADP-A2M4CD8) T cells and the endogenous unmodified T cells. The right boxplot is the density of modified (ADP-A2M4CD8) T cells only (i.e. CD3+TCR+). The plots demonstrate that tumours contain both ADP-A2M4CD8 T cells and infiltrating endogenous T cells. The closer (vertically) the right box mean horizontal line is to the left box mean horizontal line indicates a higher % of ADP-A2M4CD8 T cells out of the total CD3+ immune population.

(B) Numbers of malignant cells (PanCK+), CD4+ T cells (CD3+CD4+), cytotoxic T cells (CD3+CD8+) and regulatory T cells (CD3+CD4+FoxP3+) in in biopsies from patients with urothelial or oesophageal cancer for total, PD-L1+ and proliferating PD-L1+ (PD-L1+Ki67+) phenotypes for these cell types. Data are shown for two patients: one having urothelial cancer with stable disease (SD) clinical response to ADP-A2M4CD8 T cells; and another having oesophageal cancer with progressive disease (PD) following ADP-A2M4CD8 T cell treatment. The y-axis represents values for number of cells per mm² of tissue (density). These boxplots show changes in four different cell types post-infusion: malignant cells (PanCK+), CD4 helper T cells (CD3+CD4+), cytotoxic T cells (CD3+CD8+) and regulatory T cells (CD3+CD4+FoxP3+). Cell types are represented by the different line types (solid, dot-dash, dotted, long-dash, respectively). The solid circle symbol represents the data from the native forms of these cell types (without any PDL1+ or proliferating PDL1+Ki67+ phenotypes). For each cell type, there data are also shown for the PDL1+ phenotype (triangle symbol) and proliferating PDL1+ phenotypes (PDL1+Ki67+) (star symbol).

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a TCR” includes “TCRs”, reference to “an antibody” includes two or more such antibodies, and the like.

In general, the term “comprising” is intended to mean including but not limited to. For example, the phrase “a method comprising administering a population of modified T cells” should be interpreted to mean that the method contains a step administering such a population, but that the method may contain additional steps such as, for example, administering a further therapeutic agent.

In some aspects of the disclosure, the word “comprising” is replaced with the phrase “consisting of”. The term “consisting of” is intended to be limiting. For example, the phrase “a method consisting of administering a population of modified T cells” should be interpreted to mean that the method contains a step administering such a population, and no additional steps.

The terms “protein” and “polypeptide” are used interchangeably herein, and are intended to refer to a polymeric chain of amino acids of any length.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm.

### Treatment of Cancer

The disclosure provides a method of treating cancer in an individual, comprising (a) administering to the individual a population of modified T cells comprising a heterologous CD8 co-receptor and a heterologous TCR capable of binding to MAGE-A4; and (b) sustaining the function of (i) the population of modified T cells and/or (ii) endogenous T cells in the individual by administering an initial dose of a PD-1 axis binding antagonist to the individual about three weeks to about five weeks after step (a). A PD-1 axis binding antagonist may otherwise be known as a PD-PD-L1-PD-L2 axis inhibitor.

The disclosure also provides a population of modified T cells for use in a method of treating cancer in an individual, wherein the modified T cells comprise a heterologous CD8 co-receptor and a heterologous TCR capable of binding to MAGE-A4, and the method comprises: (a) administering the population to the individual; and (b) sustaining the function of (i) the population of modified T cells and/or (ii) endogenous T cells in the individual by administering an initial dose of a PD-1 axis binding antagonist to the individual about three weeks to about five weeks after step (a). In addition, the disclosure provides the use of a population of modified T cells in the manufacture of a medicament for use in a method of treating cancer in an individual, wherein the modified T cells comprise a heterologous CD8 co-receptor and a heterologous TCR capable of binding to MAGE-A4, and the method comprises: (a) administering the population to the individual; and (b) sustaining the function of (i) the population of modified T cells and/or (ii) endogenous T cells in the individual by administering an initial dose of a PD-1 axis binding antagonist to the individual about three weeks to about five weeks after step (a).

The disclosure further provides a PD-1 axis binding antagonist for use in a method of treating cancer in an individual, wherein the method comprises: (a) administering to the individual a population of modified T cells comprising a heterologous CD8 co-receptor and a heterologous TCR capable of binding to MAGE-A4; and (b) sustaining the function of (i) the population of modified T cells and/or (ii) endogenous T cells in the individual by administering an initial dose of the PD-1 axis binding antagonist to the individual about three weeks to about five weeks after step (a). In addition, the disclosure provides the use of a PD-1 axis binding antagonist in the manufacture of a medicament for use in a method of treating cancer in an individual, wherein the method comprises: (a) administering to the individual a population of modified T cells comprising a heterologous CD8 co-receptor and a heterologous TCR capable of binding to MAGE-A4; and (b) sustaining the function of (i) the population of modified T cells and/or (ii) endogenous T cells in the individual by administering an initial dose of the PD-1 axis binding antagonist to the individual about three weeks to about five weeks after step (a).

### Treatment of Cancer in an Individual

The disclosure concerns administration of (i) a population of modified T cells comprising a heterologous CD8 co-receptor and a heterologous TCR capable of binding to MAGE-A4 and (ii) a PD-1 axis binding antagonist in order to treat cancer in an individual. The individual may, for example, be human. The individual may, for example, be a non-human mammal such as a dog, cat or horse.

The heterologous TCR comprised in the modified T cells is capable of binding to MAGE-A4. MAGE-A4 is a well-known cancer antigen that has restricted expression in normal (i.e. non-cancerous) tissue. MAGE-A4 has been shown to repress p53 targets (such as BAX and CDKN1A) and is a binding partner for the oncogene gankyrin. The heterologous TCR may, for example, bind to GVYDGREHTV (SEQ ID NO: 1), which is a peptide sequence known as MAGE-A4230-239 that is comprised in MAGE-A4. The heterologous TCR may, for example, bind to a complex comprising MAGE-A4 (e.g. SEQ ID NO: 1) and an HLA molecule, such as HLA-A*02.

The cancer to be treated may therefore be a cancer that expresses MAGE-A4. MAGE-A4 expression has been reported for many types of cancer. The cancer may, for example, be a solid tumour. The cancer may, for example, be urothelial cancer, head and neck cancer, non-small cell lung cancer (NSCLC), oesophageal cancer, oesophogastric cancer, gastric cancer, ovarian cancer, melanoma, or endometrial cancer.

The cancer to be treated may therefore be ovarian cancer. The term “ovarian cancer” is used herein to describe cancers that begin in the cells in the ovary, fallopian tube, or peritoneum. The term “ovarian cancer” includes epithelial carcinomas; epithelial tumors include serous, endometrioid, clear cell, mucinous, mixed tumors and Brenner tumors. The term “ovarian cancer” also includes germ cell malignancies, sex cord stromal tumors and fallopian tube cancer. The term “ovarian cancer” includes primary and metastatic ovarian cancer. The term “ovarian cancer” includes ovarian cancer that has relapsed or is refractory. The term “ovarian cancer” includes ovarian cancer that may have already been subject to treatment but has recurred and/or become partially sensitive, intolerant or resistant to platinum-based therapy. The ovarian cancer may have been previously treated with surgery. The ovarian cancer may have been previously treated with radiation therapy. The ovarian cancer may have been previously treated with a chemotherapeutic agent, such as doxorubicin, docetaxel, paclitaxel, nab-paclitaxel, ifosfamide, capecitabine, fluorouracil, bleomycin, etoposide, gemcitabine, cyclophosphamide, irinotecan, melphalan, pemetrexed, vinorelbine, topotecan, vincristine, vinblastine, or dactinomycin. The ovarian cancer may have previously been treated with a platinum-based therapy, such as carboplatin, cisplatin or oxaliplatin. The ovarian cancer may have previously been treated with a targeted therapy, such as a PARP inhibitor, an anti-angiogenesis inhibitor, or a PK inhibitor; targeted therapies include bevacizumab, olaparib, niraparib, rucaparib, pazopanib, sorafenib, entrectinib, larotrectinib, trametinib, dabrafenib, vemurafenib, cobimetinib, mirvetuximab soravtansine, ofranergene obadenovec (VB-111), upifitamab rilsodotin (XMT-1536), batiraxcept (AVB-500), navicixizumab (OMP-305B83), oregovomab, nemvaleukin alfa (ALKS 4230), adavosertib (AZD1775), berzosertib (M6620, VX-970, VE-822), cediranib (AZD-2171), alpelisib (BYL719), Tumor Treating Fields (TTFields), relacorilant (CORT125134) and PC14586. The ovarian cancer may have been previously treated with hormonal therapy, such as anastrozole, exemestane, letrozole, leuprolide acetate, tamoxifen, megestrol acetate, or fulvestrant. The ovarian cancer may have been previously treated with a combination of any of the above treatments. The ovarian cancer may be recurrent after becoming intolerant or resistant to platinum-based treatment. The ovarian cancer may have progressed after one or more cycles of platinum-based treatment; for example, the ovarian cancer may have progressed after one, two, three, four, five, six, seven or eight cycles of platinum-based treatment, and wherein the ovarian cancer has progressed within about 300 days after the dose of platinum-based treatment, for example, within 14 to 300 days, within 21 to 270 days, within 30 to 240 days, within 60 to 210 days, within 90 to 195 days, after the dose of platinum-based treatment. The ovarian cancer may have progressed within 120 to 185 days after one, two or three based cycles of platinum-based treatment.

### Population of Modified T Cells

In each of the aspects of the disclosure described above, the method comprises administering to the individual a population of modified T cells comprising a heterologous CD8 co-receptor and a heterologous TCR capable of binding to MAGE-A4. It is the presence of the heterologous CD8 co-receptor and a heterologous TCR that renders the T cells “modified”. The heterologous CD8 co-receptor and the heterologous TCR are typically present on the surface of the modified T cells. In other words, the modified T cells may express the heterologous CD8 co-receptor and the heterologous TCR on their surface.

In the context of the present disclosure, the term “heterologous” refers to a polypeptide or nucleic acid that is foreign to a particular biological system (such as a T cell), i.e. that is not naturally present in that system. A “heterologous” polypeptide or nucleic acid may be introduced to the system by artificial or recombinant means. Accordingly, heterologous expression of a TCR may alter the specificity of a T cell. Heterologous expression of a CD8 co-receptor may endow the T cell with functions associated with the CD8 co-receptor. The heterologous CD8 co-receptor and the heterologous TCR are described in detail below.

The modified T cells may comprise CD4+ T cells. That is, the modified T cells may comprise T cells expressing an endogenous CD4 co-receptor. The modified T cells may comprise CD8+ T cells. That is, the modified T cells may comprise T cells expressing an endogenous CD8 co-receptor. The modified T cells may comprise CD4+ T cells and CD8+ T cells. That is, the modified T cells may comprise T cells expressing an endogenous CD4 co-receptor, and comprise T cells expressing an endogenous CD8 co-receptor. Both CD4+ T cells and CD8+ T cells are capable of harbouring a heterologous CD8 co-receptor.

The modified T cells may be allogeneic with respect to the individual. The modified T cells may preferably be autologous with respect to the individual. In this case, the modified T cells may be produced by modifying endogenous cells obtained from the individual. Thus, the method may comprise producing the population. Methods for producing modified T cells are known in the art and considered in the Example below. Typically, the modified T cells of the disclosure are produced from cells, such as peripheral blood mononuclear cells (PBMCs). T cells are typically selected from the harvested cells, and manipulated to comprise the desired modifications (here, the heterologous CD8 co-receptor and the heterologous TCR). The method may therefore comprise producing the population by: (i) obtaining peripheral blood mononuclear cells (PBMCs) from the individual; (ii) selecting T cells from the PBMCs; and (iii) modifying the selected T cells to express a heterologous CD8 co-receptor and a heterologous TCR capable of binding to MAGE-A4.

### Heterologous TCR

The modified T cells comprise a heterologous TCR capable of binding to MAGE-A4. In other words, the modified T cells express a heterologous TCR capable of binding to MAGE-A4, for instance on their surface.

The heterologous TCR may, for example be a recombinant or synthetic or artificial TCR. That is, the heterologous TCR may be a TCR that does not exist in nature. The heterologous TCR may, for example, be an affinity enhanced TCR, for example a specific peptide enhanced affinity receptor (SPEAR™) TCR.

The heterologous TCR is capable of binding to MAGE-A4. The heterologous TCR may, for example, bind to GVYDGREHTV (SEQ ID NO: 1). The heterologous TCR may, for example, bind to a complex comprising MAGE-A4 (e.g. GVYDGREHTV (SEQ ID NO: 1)) and an HLA molecule, such as HLA-A*02. In any case, the binding may be specific. Specificity refers to the strength of binding between the heterologous TCR and its target antigen. Specificity may be described by a dissociation constant, Kd, the ratio between bound and unbound states for the receptor-ligand system. Typically, the fewer different antigens the heterologous TCR is capable of binding other than MAGE-A4, the greater its binding specificity.

The heterologous TCR may, for example, bind to MAGE-A4 with a dissociation constant (Kd) of between 0.01 μM and 100 μM, between 0.01 μM and 50 μM, between 0.01 μM and 20 μM, between 10 μM and 1000 μM, between 10 μM and 500 μM, or between 50 μM and 500 μM. For instance, in a preferred aspect of the disclosure, the heterologous TCR binds to MAGE-A4 with a Kd of between 0.05 μM to 20.0 μM. For example, the heterologous TCR may bind to MAGE-A4 with a Kd of 0.01 μM, 0.02 μM, 0.03 μM, 0.04 μM, 0.05 μM, 0.06 μM, 0.07 μM, 0.08 μM, 0.09 μM, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10.0 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM. The Kd may, for example, be measured using surface plasmon resonance, optionally at 25° C., optionally between a pH of 6.5 and 6.9 or 7.0 and 7.5. The dissociation constant, Kd or koff/kon may be determined by experimentally measuring the dissociation rate constant, koff, and the association rate constant, kon. A TCR dissociation constant may be measured using a soluble form of the TCR, wherein the TCR comprises a TCR alpha chain variable domain and a TCR beta chain variable domain.

The heterologous TCR may, for example, comprise an alpha chain variable domain having at least 80% sequence identity to the sequence of amino acid residues 22-125 of SEQ ID NO: 2. The heterologous TCR may, for example, comprise a beta chain variable domain having at least 80% sequence identity to the sequence of amino acid residues 22-123 of SEQ ID NO: 3. The heterologous TCR may, for example, comprise an alpha chain variable domain having at least 80% sequence identity to the sequence of amino acid residues 22-125 of SEQ ID NO: 2 and a beta chain variable domain having at least 80% sequence identity to the sequence of amino acid residues 22-123 of SEQ ID NO: 3. The alpha chain variable domain may, for example, have at least 85%, at least 90%, at least 95%, at least 97%, at least 98% or at least 99% sequence identity to the sequence of amino acid residues 22-125 of SEQ ID NO: 2. The alpha chain variable domain may, for example, comprise or consist of amino acid residues 22-125 of SEQ ID NO: 2. The beta chain variable domain may, for example, have at least 85%, at least 90%, at least 95%, at least 97%, at least 98% or at least 99% sequence identity to the sequence of amino acid residues 22-123 of SEQ ID NO: 3. The beta chain amino acid sequence may, for example, comprise or consist of amino acid residues 22-123 of SEQ ID NO: 3.

The heterologous TCR may, for example, comprise an alpha chain having at least 80% sequence identity to the sequence of amino acid residues 22-282 of SEQ ID NO: 2. The heterologous TCR may, for example, comprise a beta chain having at least 80% sequence identity to the sequence of amino acid residues 22-311 of SEQ ID NO: 3. The heterologous TCR may, for example, comprise an alpha chain having at least 80% sequence identity to the sequence of amino acid residues 22-282 of SEQ ID NO: 2 and a beta chain variable domain having at least 80% sequence identity to the sequence of amino acid residues 22-311 of SEQ ID NO: 3. The alpha chain variable domain may, for example, have at least 85%, at least 90%, at least 95%, at least 97%, at least 98% or at least 99% sequence identity to the sequence of amino acid residues 22-282 of SEQ ID NO: 2. The alpha chain variable domain may, for example, comprise or consist of amino acid residues 22-282 of SEQ ID NO: 2. The beta chain variable domain may, for example, have at least 85%, at least 90%, at least 95%, at least 97%, at least 98% or at least 99% sequence identity to the sequence of amino acid residues 22-311 of SEQ ID NO: 3. The beta chain amino acid sequence may, for example, comprise or consist of amino acid residues 22-311 of SEQ ID NO: 3.

The heterologous TCR is typically expressed with N-terminal signal peptides that are cleaved prior to expression at the surface of the T cell. In this respect, amino acids 1 to 21 of each of SEQ ID NO: 2 and SEQ ID NO: 3 are typically cleaved prior to expression of the TCR at the surface of the T cell. The heterologous TCR may, for example, comprise an alpha chain amino acid sequence having at least 80% sequence identity to SEQ ID NO: 2. The heterologous TCR may, for example, comprise a beta chain amino acid sequence having at least 80% sequence identity to SEQ ID NO: 3. The heterologous TCR may, for example, comprise an alpha chain amino acid sequence having at least 80% sequence identity to SEQ ID NO: 2 and a beta chain amino acid sequence having at least 80% sequence identity to SEQ ID NO: 3. The alpha chain amino acid sequence may, for example, have at least 85%, at least 90%, at least 95%, at least 97%, at least 98% or at least 99% sequence identity to SEQ ID NO: 2. The alpha chain amino acid sequence may, for example, comprise or consist of SEQ ID NO: 2. The beta chain amino acid sequence may, for example, have at least 85%, at least 90%, at least 95%, at least 97%, at least 98% or at least 99% sequence identity to SEQ ID NO: 3. The beta chain amino acid sequence may, for example, comprise or consist of SEQ ID NO: 3.

The heterologous TCR may, for example, comprise (i) an alpha chain variable domain that comprises a CDR1 that comprises (1) the sequence of SEQ ID NO: 4 or (2) an amino acid sequence that comprises one, two or three amino acid insertions, deletions or substitutions relative to the sequence of SEQ ID NO: 4; (ii) an alpha chain variable domain that comprises a CDR2 that comprises (1) the sequence of SEQ ID NO: 5 or (2) an amino acid sequence that comprises one, two or three amino acid insertions, deletions or substitutions relative to the sequence of SEQ ID NO: 5; (iii) an alpha chain variable domain that comprises a CDR3 that comprises (1) the sequence of SEQ ID NO: 6 or (2) an amino acid sequence that comprises one, two or three amino acid insertions, deletions or substitutions relative to the sequence of SEQ ID NO: 6; (iv) a beta chain variable domain that comprises a CDR1 that comprises (1) the sequence of SEQ ID NO: 7 or (2) an amino acid sequence that comprises one, two or three amino acid insertions, deletions or substitutions relative to the sequence of SEQ ID NO:7; (v) a beta chain variable domain that comprises a CDR2 that comprises (1) the sequence of SEQ ID NO: 8 or (2) an amino acid sequence that comprises one, two or three amino acid insertions, deletions or substitutions relative to the sequence of SEQ ID NO: 8; and/or (vi) a beta chain variable domain that comprises a CDR3 that comprises (1) the sequence of SEQ ID NO: 9 or (2) an amino acid sequence that comprises one, two or three amino acid insertions, deletions or substitutions relative to the sequence of SEQ ID NO: 9.

The alpha chain of the heterologous TCR may, for example, comprise (i) an alpha chain variable domain that comprises a CDR1 that comprises (1) the sequence of SEQ ID NO: 4 or (2) an amino acid sequence that comprises one, two or three amino acid insertions, deletions or substitutions relative to the sequence of SEQ ID NO: 4; (ii) an alpha chain variable domain that comprises a CDR2 that comprises (1) the sequence of SEQ ID NO: 5 or (2) an amino acid sequence that comprises one, two or three amino acid insertions, deletions or substitutions relative to the sequence of SEQ ID NO: 5; and (iii) an alpha chain variable domain that comprises a CDR3 that comprises (1) the sequence of SEQ ID NO: 6 or (2) an amino acid sequence that comprises one, two or three amino acid insertions, deletions or substitutions relative to the sequence of SEQ ID NO: 6. The alpha chain of the heterologous TCR may, for example, comprise (i) an alpha chain variable domain that comprises a CDR1 that comprises the sequence of SEQ ID NO: 4; (ii) an alpha chain variable domain that comprises a CDR2 that comprises the sequence of SEQ ID NO: 5; and (iii) an alpha chain variable domain that comprises a CDR3 that comprises the sequence of SEQ ID NO: 6.

The beta chain of the heterologous TCR may, for example, comprise (iv) a beta chain variable domain that comprises a CDR1 that comprises (1) the sequence of SEQ ID NO: 7 or (2) an amino acid sequence that comprises one, two or three amino acid insertions, deletions or substitutions relative to the sequence of SEQ ID NO:7; (v) a beta chain variable domain that comprises a CDR2 that comprises (1) the sequence of SEQ ID NO: 8 or (2) an amino acid sequence that comprises one, two or three amino acid insertions, deletions or substitutions relative to the sequence of SEQ ID NO: 8; and (vi) a beta chain variable domain that comprises a CDR3 that comprises (1) the sequence of SEQ ID NO: 9 or (2) an amino acid sequence that comprises one, two or three amino acid insertions, deletions or substitutions relative to the sequence of SEQ ID NO: 9. The beta chain of the heterologous TCR may, for example, comprise (iv) a beta chain variable domain that comprises a CDR1 that comprises the sequence of SEQ ID NO: 7; (v) a beta chain variable domain that comprises a CDR2 that comprises the sequence of SEQ ID NO: 8; and (vi) a beta chain variable domain that comprises a CDR3 that comprises the sequence of SEQ ID NO: 9.

The heterologous TCR may, for example, comprise an alpha chain comprising a CDR1 having the sequence of SEQ ID NO: 4, a CDR2 having the sequence of SEQ ID NO: 5 and a CDR3 having the sequence of SEQ ID NO: 6, and a beta chain comprising a CDR1 having the sequence of SEQ ID NO: 7, a CDR2 having the sequence of SEQ ID NO: 8 and a CDR3 having the sequence of SEQ ID NO: 9. The heterologous TCR may, for example, have additionally any of the percentage identities in the alpha chain and beta chain discussed herein.

### Heterologous CD8 Co-Receptor

The modified T cells comprise a heterologous CD8 co-receptor. In other words, the modified T cells express a heterologous CD8 co-receptor, for instance on their surface.

CD8 is a cell surface glycoprotein that, in nature, is found on most cytotoxic T lymphocytes and mediates efficient cell-cell interactions within the immune system. CD8 acts as a co-receptor for the T cell receptor, such that CD8 and the T cell receptor together recognise antigen displayed by an antigen-presenting cell in the context of class I MHC molecules. The CD8 co-receptor binds to class 1 MHCs and potentiates TCR signaling. The functional co-receptor may be a homodimer consisting of two CD8 alpha chains, or a heterodimer consisting of one CD8 alpha chain and one CD8 beta chain.

Accordingly, the heterologous CD8 co-receptor comprised in the modified T cells may be CD8α. In other words, the heterologous CD8 co-receptor may be a homodimer consisting of two CD8 alpha chains. Alternatively, the heterologous CD8 co-receptor may be a heterodimer consisting of one CD8 alpha chain and one CD8 beta chain. In either case, a CD8 alpha chain may comprise or consist of an amino acid sequence having at least 80% (such as at least 85%, at least 90%, at least 95%, at least 97%, at least 98%, at least 99% or 100%) sequence identity to SEQ ID NO: 10. Thus, the heterologous CD8 co-receptor may comprise an amino acid sequence having at least 80% (such as at least 85%, at least 90%, at least 95%, at least 97%, at least 98%, at least 99% or 100%) sequence identity to SEQ ID NO: 10.

CD8 alpha chains and CD8 beta chains both share significant homology to immunoglobulin variable light chains. The heterologous CD8 co-receptor may, for example, comprise a CD8 alpha chain that comprises: (i) an alpha chain CDR1 that comprises (1) the sequence of SEQ ID NO: 11 or (2) an amino acid sequence that comprises one, two or three amino acid insertions, deletions or substitutions relative to the sequence of SEQ ID NO: 11; (ii) an alpha chain CDR2 that comprises (1) the sequence of SEQ ID NO: 12 or (2) an amino acid sequence that comprises one, two or three amino acid insertions, deletions or substitutions relative to the sequence of SEQ ID NO: 12; and/or (iii) an alpha chain CDR3 that comprises (1) the sequence of SEQ ID NO: 13 or (2) an amino acid sequence that comprises one, two or three amino acid insertions, deletions or substitutions relative to the sequence of SEQ ID NO: 13.

The heterologous CD8 co-receptor is capable of binding to a class I MHC molecule. The heterologous CD8 co-receptor may, for example, bind to the α₃ portion of a class I MHC molecule, for instance via the IgV-like domain of the CD8 co-receptor. The α₃ portion is typically found between residues 223 and 229 of a class I MHC molecule. The ability of the heterologous CD8 co-receptor to bind to a class I MHC molecule improves the ability of the modified T cells to engage cognate antigen via their heterologous TCR. The cognate antigen, MAGE-A4, is typically presented in complex with a class I MHC molecule such as HLA-A*02. The heterologous CD8 co-receptor may improve or increase the off-rate (k_{off}) of the TCR/peptide-MHCI interaction in the modified cells. The improvement or increase may be relative to modified T cells that comprise a heterologous TCR that binds to MAGE-A4 but which lack a heterologous CD8 co-receptor. The heterologous CD8 co-receptor may, for example, assist in organising the heterologous TCR on the surface of modified cells, thereby improving the ability of the heterologous TCR to participate in the TCR/peptide-MHCI interaction. The heterologous CD8 co-receptor may, for example, bind or interact with LCK (lymphocyte-specific protein tyrosine kinase) in a zinc-dependent manner leading to activation of transcription factors like NFAT, NF-κB, and AP-1.

Accordingly, expression of a heterologous CD8 co-receptor may confer upon the modified T cells an improved affinity and/or avidity for MAGE-A4, and/or improved activation upon binding to MAGE-A4. Methods for determining affinity, avidity and T cell activation are well-known in the art. Expression of a heterologous CD8 co-receptor may confer upon the modified T cells an improved or increased expression of CD40L, cytokine production, cytotoxic activity, induction of dendritic cell maturation or induction of dendritic cell cytokine production, for instance in response to antigen (MAGE-A4) binding. Improvements or increases may be relative to modified T cells that comprise a heterologous TCR that binds to MAGE-A4 but which lack a heterologous CD8 co-receptor.

Synergy has been demonstrated between CD8α and peptide antigen presented on HLA-A*0201. Therefore, in one aspect of the disclosure, the heterologous CD8 co-receptor may be CD8α and the heterologous TCR may be capable of binding to a peptide antigen of MAGE-A4 in complex with HLA-A*0201. The peptide antigen may, for example, be SEQ ID NO: 1.

### PD-1 Axis Binding Antagonist

Programmed cell death protein 1 (PD-1, also known as CD279) is a protein that is expressed on the surface of T cells and has a role in regulating immune responses by maintaining T cell homeostasis. Ligation of PD-1 to one of its ligands (PD-L1 or PD-L2) transmits an inhibitory signal within the T cell. In particular, PD-1-generated signals prevent phosphorylation of key TCR signalling intermediates, thereby terminating early TCR signalling and reducing T cell activation. T cell effector functions (such as proliferation, cytotoxicity and cytokine production) are reduced, and the ability to transition to memory T cells is impaired.

PD-L1 and PD-L2 are members of the B7 family. PD-L1 protein is upregulated on certain activated immune cells (such as macrophages, dendritic cells, T cells and B cells), and is also expressed upon certain normal tissues. PD-L1 is also highly expressed in many cancers. PD-L2 is expressed mainly by dendritic cells and some tumours. As many cancers express PD-1 ligands, the PD-1 axis has an established role in cancer immune evasion and tumour resistance.

In the present disclosure, the method comprises sustaining the function of (i) the population of modified T cells and/or (ii) endogenous T cells in the individual by administering a PD-1 axis binding antagonist to the individual. Expression of PD-1 ligands by cancers, such as solid tumours, renders the tumour microenvironment immunosuppressive. The function of modified T cells infiltrating the tumour may therefore be inhibited. Endogenous anti-tumour T cell responses may also be inhibited. In this way, tumours are more able to evade the immune system. In the present disclosure, a PD-1 axis binding antagonist is administered to counteract suppressive effects of PD-L1 and/or PD-L2 expression in the tumour microenvironment. By counteracting suppression, the function of modified and/or endogenous T cells may be sustained. That is, administration of a PD-1 axis binding antagonist may sustain the function of modified T cells comprised in the administration, and/or their descendants. Administration of a PD-1 axis binding antagonist may sustain the function of endogenous T cells in the individual. Preferably, administration of a PD-1 axis binding antagonist sustains the function of modified T cells comprised in the administration (and/or their descendants), and of endogenous T cells in the individual. In any case, the endogenous T cells may, for example, be comprised in the tumour microenvironment. For instance, the endogenous T cells may be tumour infiltrating lymphocytes (TILs).

Sustaining the function of T cells may refer, for example, to maintaining, restoring and/or enhancing T cell function. Sustaining T cell function may, for example, refer to sustaining T cell activation. In this way, the duration of an effective T cell response may be extended. In other words, sustained activation maybe associated with an improved duration of effector function (such as cytokine production, cytotoxicity and/or proliferation). Sustained activation may also assist the T cells' ability to transition to memory T cells. The generation of memory T cells is advantageous, as it permits anti-tumour immunity to be maintained in the long-term e.g. for months or years. Methods for determining activation, cytokine production, cytotoxicity, proliferation, and generation of memory T cells are well-known in the art.

Administration of the PD-1 axis binding antagonist may sustain the function of the modified T cells and/or endogenous T cells by reducing exhaustion. Exhaustion may be reduced within the population of modified T cells, and/or within T cells descended from the population of modified T cells. Exhaustion may be reduced within endogenous T cells in the individual. Exhaustion may be reduced (i) within the population of modified T cells and/or within T cells descended from the population of modified T cells, and (ii) within endogenous T cells in the individual. Exhausted T cells typically express high levels of PD-1, and experience a loss of function. For instance, exhausted T cells may have reduced ability to produce cytokines such as IL-2 or TNFα. Exhausted T cells may have reduced proliferative capacity. Exhausted T cells may have reduced cytotoxic potential. Ultimately, exhausted T cells may be targeted for destruction. Exhaustion therefore causes loss of T cell function, or loss of T cells themselves, which is disadvantageous to tumour immunity. Reducing T cell exhaustion may therefore improve therapeutic outcome.

In the context of the disclosure, a PD-1 axis binding antagonist is a molecule that inhibits the interaction of PD-1 with a PD-1 ligand, and/or transduction of a signal resulting from the interaction of PD-1 with a PD-1 ligand. The PD-1 ligand may be PD-L1 or PD-L2. The PD-1 axis binding antagonist may, for example, reduce or prevent the interaction of PD-1 with a PD-1 ligand. The PD-1 axis binding antagonist may, for example, reduce or prevent transduction of a signal resulting from the interaction of PD-1 with a PD-1 ligand. The PD-1 axis binding antagonist may block, inhibit or reduce the biological activity of PD-1 and/or a PD-1 ligand.

By inhibiting the interaction of PD-1 with a PD-1 ligand, and/or transduction of a signal resulting from the interaction of PD-1 with a PD-1 ligand, the PD-1 axis binding antagonist may sustain (e.g. maintain, restore or enhance) the function of endogenous T cells. In the same way, the PD-1 axis binding antagonist may sustain (e.g. maintain, restore or enhance) the function of modified T cells administered to the individual. Sustained function may, for example, be indicated by maintenance of, or improvements in, T-cell proliferation, cytokine production, target cell killing, activation, CD28 signalling, ability to infiltrate tumour, ability to recognise and bind to dendritic cell presented antigen, and/or ability to produce interferon. In this way, the PD-1 axis binding antagonist counteracts the immunosuppressive nature of the tumour microenvironment.

The PD-1 axis binding antagonist may, for example, be a PD-1 binding antagonist. That is, the PD-1 axis binding antagonist may inhibit (e.g. prevent or reduce) binding of PD-1 to its binding partners. For example, the PD-1 axis binding antagonist may inhibit the binding of PD-1 to PD-L1, PD-L2, or both PD-L1 and PD-L2. The PD-1 binding antagonist may, for example, be an antibody that binds to PD-1, or an antigen-binding variant or fragment thereof. The PD-1 binding antagonist may, for example, be an antibody that binds to SEQ ID NO: 14, or an antigen-binding variant or fragment thereof. Antibodies that bind to PD-1 are well-known in the art and include, for example, nivolumab, pembrolizumab, cemiplimab, dostarlimab, JTX-4014, spartalizumab, camrelizumab, sintilimab, tislelizumab, toripalimab, retifanlimab (INCMGA00012), AMP-224, MEDI0680 (AMP-514), sasanlimab, budigalimab, ezabenlimab (BI 754091), zimberelimab (AB122). The PD-1 axis binding antagonist may therefore be nivolumab, pembrolizumab, cemiplimab, dostarlimab, JTX-4014, spartalizumab, camrelizumab, sintilimab, tislelizumab, toripalimab, retifanlimab (INCMGA00012), AMP-224, MEDI0680 (AMP-514), sasanlimab, budigalimab, ezabenlimab (BI 754091), zimberelimab (AB122) or any combination thereof. The PD-1 axis binding antagonist may, for example, be nivolumab. The PD-1 binding antagonist may alternatively be any immunoadhesin, protein or oligopeptide that inhibits, decreases, prevent, or interferes with signal transduction due to the interaction of PD-1 with PD-L1 and/or PD-L2.

The heavy chain sequence and light chain sequence of nivolumab are set out in SEQ ID NOs: 17 and 18 respectively. The heavy chain sequence and light chain sequence of pembrolizumab are set out in SEQ ID NOs: 19 and 20 respectively. The heavy chain sequence and light chain sequence of cemiplimab are set out in SEQ ID NOs: 21 and 22 respectively. The heavy chain sequence and light chain sequence of dostarlimab are set out in SEQ ID NOs: 31 and 32 respectively. A skilled person equipped with the heavy and light chain sequences of a given antibody may identify antigen-binding variants or fragments of the antibody using methods routine in the art. An antigen-binding variant or fragment of nivolumab may, for example, comprise the three CDRs comprised in SEQ ID NO: 17 and the three CDRs comprised in SEQ ID NO: 18. An antigen-binding variant or fragment of pembrolizumab may, for example, comprise the three CDRs comprised in SEQ ID NO: 19 and the three CDRs comprised in SEQ ID NO: 20. An antigen-binding variant or fragment of cemiplimab may, for example, comprise the three CDRs comprised in SEQ ID NO: 21 and the three CDRs comprised in SEQ ID NO: 22. An antigen-binding variant or fragment of dostarlimab may, for example, comprise the three CDRs comprised in SEQ ID NO: 31 and the three CDRs comprised in SEQ ID NO: 32. Methods for identifying CDRs within a heavy chain or light chain sequence are routine in the art.

The PD-1 axis binding antagonist may, for example, be a PD-L1 binding antagonist. That is, the PD-1 axis binding antagonist may inhibit (e.g. prevent or reduce) binding of PD-L1 to a binding partner. For example, the PD-1 axis binding antagonist may inhibit the binding of PD-L1 to PD-1. The PD-L1 binding antagonist may, for example, be an antibody that binds PD-L1, or an antigen-binding variant or fragment thereof. The PD-L1 binding antagonist may, for example, be an antibody that binds to SEQ ID NO: 15, or an antigen-binding variant or fragment thereof. Antibodies that bind to PD-L1 are well-known in the art and include, for example, durvalumab, atezolizumab, avelumab, BMS 936559 (MDX-1105), envafolimab (KN035) and cosibelimab (CK-301). The PD-L1 axis binding antagonist may therefore be durvalumab, atezolizumab, avelumab, BMS 936559 (MDX-1105), envafolimab (KN035), cosibelimab (CK-301), or any combination thereof. The PD-L1 binding antagonist may alternatively be any immunoadhesin, protein or oligopeptide that inhibits, decreases, prevent, or interferes with signal transduction due to the interaction of PD-L1 with PD-1.

The heavy chain sequence and light chain sequence of durvalumab are set out in SEQ ID NOs: 23 and 24 respectively. The heavy chain sequence and light chain sequence of atezolizumab are set out in SEQ ID NOs: 25 and 26 respectively. The heavy chain sequence and light chain sequence of avelumab are set out in SEQ ID NOs: 27 and 28 respectively. The heavy chain sequence and light chain sequence of BMS 936559 (MDX-1105) are set out in SEQ ID NOs: 29 and 30 respectively. A skilled person equipped with the heavy and light chain sequences of a given antibody may identify antigen-binding variants or fragments of the antibody using methods routine in the art. An antigen-binding variant or fragment of durvalumab may, for example, comprise the three CDRs comprised in SEQ ID NO: 23 and the three CDRs comprised in SEQ ID NO: 24. An antigen-binding variant or fragment of atezolizumab may, for example, comprise the three CDRs comprised in SEQ ID NO: 25 and the three CDRs comprised in SEQ ID NO: 26. An antigen-binding variant or fragment of avelumab may, for example, comprise the three CDRs comprised in SEQ ID NO: 27 and the three CDRs comprised in SEQ ID NO: 28. An antigen-binding variant or fragment of BMS 936559 (MDX-1105) may, for example, comprise the three CDRs comprised in SEQ ID NO: 29 and the three CDRs comprised in SEQ ID NO: 30. Methods for identifying CDRs within a heavy chain or light chain sequence are routine in the art.

The PD-1 axis binding antagonist may, for example, be a PD-L2 binding antagonist. That is, the PD-1 axis binding antagonist may inhibit (e.g. prevent or reduce) binding of PD-L2 to a binding partner. The PD-L2 binding antagonist may, for example, be an antibody that binds PD-L2, or an antigen-binding variant or fragment thereof. The PD-L2 binding antagonist may, for example, be an antibody that binds to SEQ ID NO: 16, or an antigen-binding variant or fragment thereof. The PD-L2 binding antagonist may alternatively be any immunoadhesin, protein or oligopeptide that inhibits, decreases, prevent, or interferes with signal transduction due to the interaction of PD-L2 with PD-1.

When the PD-1 axis binding antagonist is an antibody (such as a known antibody, or an antigen-binding variant thereof), the PD-1 axis binding antagonist may be a monoclonal antibody, a human or humanised antibody, a full-length antibody, a diabody, a linear antibody, or a single-chain antibody molecule, for example. The antibody isotype may be selected from any of the five classes of immunoglobulins: IgA, IgD, IgE, IgG and IgM, having heavy chains designated alpha, delta, epsilon, gamma and mu (M), respectively. The gamma and alpha class antibodies may be of any of subclasses: IgG1, IgG2A, IgG2B, IgG3, IgG4, IgA1 and IgA2. When the PD-1 axis binding antagonist is an antigen-binding fragment of an antibody, the PD-1 axis binding antagonist may be a Fv, Fab, Fab′, Fab′-SH, F(ab′)2, or scFv, for example.

When the PD-1 axis binding antagonist is an immunoadhesin, the immunoadhesin may comprise an adhesin domain conferring binding activity for a PD-1 axis component (e.g. PD-1, PD-L1, or PD-L2) and an immunoglobulin constant domain. The immunoglobulin constant domain may be from any isotype, such as IgG1, IgG2, IgG2A, IgG2B, IgG3, IgG4 subtypes, IgA, IgA1, IgA2, IgE, IgD or IgM. The immunoglobulin constant domain may, for example, comprise (i) the hinge, CH2 and CH3, or (ii) the hinge, CH1, CH2 and CH3 regions of an immunoglobulin molecule. Accordingly, the immunoadhesin may comprise (a) the extracellular or PD-1 binding portions of PD-L1 or PD-L2, or the extracellular or PD-L1 or PD-L2 binding portions of PD-1, fused to (b) a constant domain of an immunoglobulin sequence.

### Administration

In the present disclosure, the method comprises (a) administering the population of modified T cells to the individual; and (b) sustaining the function of (i) the population of modified T cells and/or (ii) endogenous T cells in the individual by administering an initial dose of the PD-1 axis binding antagonist.

The population may comprise any number of modified T cells that will be therapeutically effective. The number of modified T cells for a given individual may depend on factors such as the cancer to be treated, the severity or stage of the cancer, the age of the patient and so on. The number to be administered may thus depend on the judgement of the practitioner and may be peculiar to each subject. By way of example, the population may comprise about 0.8×10⁹ to about 10×10⁹ modified T cells, such as about 0.8×10⁹ to about 1.2×10⁹ modified T cells, about 1.2×10⁹ to about 6×10⁹ modified T cells, or about 1.0×10⁹ to 10×10⁹ modified T cells. The population may, for example, comprise 1.0×10⁹ modified T cells, about 5.0×10⁹ modified T cells, or about 10×10⁹ modified T cells.

Typically, the population of modified T cells is administered as a single dose. One or more (such as two or more, three or more, four or more, or five or more) further doses may though be administered depending on patient factors and the judgement of the practitioner.

Typically, the population of modified T cells is administered intravenously. Any suitable route may though be used, for instance intramuscular, subcutaneous, intradermal, transdermal, or intraperitoneal routes.

The initial dose of the PD-1 axis binding antagonist is administered about three weeks to about five weeks after administration of the population of modified T cells. The initial dose of the PD-1 axis binding antagonist may, for example, be administered about three weeks, about four weeks, or about five weeks after administration of the population of modified T cells. The initial dose of the PD-1 axis binding antagonist may, for example, be administered about 21 days to about 35 days after administration of the population of modified T cells, such as about 25 days to about 30 days. The initial dose of the PD-1 axis binding antagonist may, for example, be administered about 21 days, about 22 days, about 23 days, about 24 days, about 25 days, about 26 days, about 27 days, about 28 days, about 29 days, about 30 days, about 31 days, about 32 days, about 33 days, about 34 days or about 35 days after administration of the population of modified T cells. Preferably, the initial dose of the PD-1 axis binding antagonist is administered about 4 weeks (i.e. about 28 days) after administration of the population of modified T cells. For instance, the initial dose of the PD-1 axis binding antagonist may be administered about 25 days to about 30 days (such as about 25 days, about 26 days, about 27 days, about 28 days, about 29 days, or about 30 days) after administration of the population of modified T cells.

The present inventors have identified that this window between administration of the population of modified T cells and administration of initial dose of the PD-1 axis binding antagonist is, surprisingly, advantageous. As demonstrated in the Example, the inventors have demonstrated that modified T cells peak at around 13 to 16 days post-administration and persist at optimal levels for more than 24 weeks. In responsive individuals (as determined by Best Objective Response, BOR), this unexpectedly early peak in T cell persistence is followed by a reduction in the diameters of target lesions at the earliest studied time point, four weeks post-administration. This indicates that the modified T cells have a therapeutic effect at a surprisingly early time point. Furthermore, the Example demonstrates that TILs exist in target tumours by week four after administration of modified T cells, and that TILs comprise both modified T cells and T cells endogenous to the individual. The inventors have therefore shown that a PD-PD-L1-PD-L2 axis inhibitor may optimally be administered at or around four weeks after infusion of modified T cells. Data indicate that, at this time point, modified and endogenous T cells have infiltrated the tumour and are mounting an effective anti-tumour response. Administration of a PD-PD-L1-PD-L2 axis inhibitor at or around four weeks post-infusion coincides with strong T cell activity/functionality, and thus helps to sustain T cell activity/functionality, by delaying T cell exhaustion mediated by PD-PD-L1-PD-L2 axis signalling. In this way, effector function may be prolonged and the durability of activated T cells increased.

Timing administration of initial dose of the PD-1 axis binding antagonist to coincide with the presence of TILs and/or the onset of a therapeutic effect of the modified T cells is beneficial, as it allows activation (and thus function) of adoptively-transferred and endogenous T cells to be maintained at therapeutically-effective levels. It also encourages the generation of memory T cells, thereby affording long-lived anti-tumour immunity and thus improving outcomes for patients.

The method may, for example, comprise administering one or more further doses of the PD-1 axis binding antagonist. The purpose of the one or more further doses of the PD-1 axis binding antagonist may be to maintain the effects achieved by administration of the initial dose. Each of the one or more further doses may comprise the same PD-1 axis binding antagonist as the initial dose, or a different PD-1 axis binding antagonist from the initial dose.

The one or more further doses of the PD-1 axis binding antagonist may be administered at any appropriate interval. Suitable dosage intervals for PD-1 axis binding antagonists are known in the art. The one or more further doses may, for example, be administered about once every two weeks (Q2W) beginning two weeks from administration of the initial dose. The one or more further doses may, for example, be administered about once every three weeks (Q3W) beginning three weeks from administration of the initial dose. The one or more further doses may, for example, be administered about once every four weeks (Q4W) beginning four weeks from administration of the initial dose. The one or more further doses may, for example, be administered about once every five weeks (Q5W) beginning five weeks from administration of the initial dose. The one or more further doses may, for example, be administered about once every six weeks (Q6W) beginning six weeks from administration of the initial dose. In a preferred aspect of the disclosure, the one or more further doses is administered about once every four weeks (Q4W) beginning four weeks from administration of the initial dose. Any number of further doses may be administered, such as one or more, two or more, three or more, four or more, five or more, six or more, seven or more, eight or more, nine or more, ten or more, 15 or more, 20 or more, 25 or more, 30 or more, 35 or more, 40 or more, 45 or more or 50 or more further doses. Further doses may be administered until disease progression, unacceptable toxicity, withdrawal of consent, or death.

The initial dose of the PD-1 axis binding antagonist, and/or any further dose of the PD-1 axis binding antagonist, may comprise any therapeutically effective amount of the PD-1 axis binding antagonist. The amount for a given individual may depend on factors such as the cancer to be treated, the severity or stage of the cancer, the age of the patient and so on. The number to be administered may thus depend on the judgement of the practitioner and may be peculiar to each subject. By way of example, the initial dose of the PD-1 axis binding antagonist, and/or any further dose of the PD-1 axis binding antagonist, may comprise about 200 mg to about 700 mg of the PD-1 axis binding antagonist, such as about 300 mg to about 600 mg, about 400 mg to about 500 mg, or about 450 mg to 500 mg of the PD-1 axis binding antagonist. For instance, the initial dose of the PD-1 axis binding antagonist, and/or any further dose of the PD-1 axis binding antagonist, may comprise about 200 mg, about 250 mg, about 300 mg, about 350 mg, about 400 mg, about 450 mg, about 500 mg, about 550 mg, about 600 mg, about 650 mg, or about 700 mg of the PD-1 axis binding antagonist. The initial dose of the PD-1 axis binding antagonist, and/or any further dose of the PD-1 axis binding antagonist, may, for example, comprise about 450 mg to about 500 mg of the PD-1 axis binding antagonist, such as about 480 mg of the PD-1 axis binding antagonist. In a preferred aspect of the disclosure, the initial dose of the PD-1 axis binding antagonist, and any further dose of the PD-1 axis binding antagonist, comprises 480 mg of nivolumab.

By way of example, the PD-1 axis binding antagonist may, for example, be administered at an initial dose of about 480 mg, about four weeks after administration of the population of modified T cells. One or more further does may be administered about every four weeks beginning four weeks from administration of the initial dose. Each further dose may comprise about 480 mg of the PD-1 axis binding antagonist. The PD-1 axis binding antagonist may, for example, be nivolumab.

In another example, the PD-1 axis binding antagonist may, for example, be administered at an initial dose of about 240 mg, about four weeks after administration of the population of modified T cells. One or more further does may be administered about every two weeks beginning two weeks from administration of the initial dose. Each further dose may comprise about 240 mg of the PD-1 axis binding antagonist. The PD-1 axis binding antagonist may, for example, be nivolumab.

Typically, the PD-1 axis binding antagonist is administered intravenously. Any suitable route may though be used, for instance intramuscular, subcutaneous, intradermal, transdermal, or intraperitoneal routes.

In some aspects of the disclosure, the method comprises administering lymphodepleting chemotherapy to the individual prior to administration of the population of modified T cells. That is, lymphodepleting chemotherapy may be administered before step (a). Lymphodepleting chemotherapy may, for example, be administered from about 14 days before step (a) to about 1 day before step (a), such as about 13 days before step (a) to about 2 days before step (a), about 12 days before step (a) to about 3 days before step (a), about 11 days before step (a) to about 4 days before step (a), about 10 days before step (a) to about 5 days before step (a), about 9 days before step (a) to about 6 days before step (a), about 8 days before step (a) to about 7 days before step (a), about 10 days before step (a) to about 1 day before step (a), about 9 days before step (a) to about 2 days before step (a), about 8 days before step (a) to about 3 days before step (a), about 7 days before step (a) to about 4 days before step (a), or about 6 days before step (a) to about 5 days before step (a). Preferably, lymphodepleting chemotherapy is administered from about 7 days before step (a) to about 4 days before step (a). The purpose of lymphodepleting chemotherapy may be to deplete the lymphocyte compartment of the individual, so as to provide space into which the adoptively-transferred modified T cells can expand. In this way, the effects of a given dose of the modified T cells can be maximised. The lymphodepleting chemotherapy may comprise any suitable lymphotoxic agent. Lymphotoxic agents and suitable dosages are known in the art. The lymphodepleting chemotherapy may, for example, comprise fludarabine and/or cyclophosphamide. Typically, the lymphodepleting chemotherapy is administered intravenously. Any suitable route may though be used, for instance intramuscular, subcutaneous, intradermal, transdermal, or intraperitoneal routes.

### EXAMPLE

### Dosage Regime for ADP-A2M4CD8 with PD-1 Axis Binding Antagonist

### Introduction

ADP-A2M4CD8 specific peptide enhanced affinity receptor (SPEAR™) T cells are genetically engineered to target the tumour antigen MAGE-A4 in the context of the appropriate human leukocyte antigen (HLA) expression. ADP-A2M4CD8 are autologous CD4 and CD8 positive T cells that have been transduced with a self-inactivating (SIN) lentiviral vector expressing a high affinity MAGE-A4 specific T cell receptor (TCR) and an additional CD8α co-receptor.

The affinity-optimised TCR (ADP-A2M4 TCR) comprises an alpha chain variable domain having the amino acid sequence of SEQ ID NO: 2, and a beta chain variable domain having the amino acid sequence of SEQ ID NO: 3. When expressed in a T cell, the signal peptides are cleaved from SEQ ID NOs: 2 and 3 prior to surface expression. A2M4 TCR targets the tumour antigen MAGE-A4 and activates engineered T cells. It recognizes the MAGE-A4230-239 (GVYDGREHTV; SEQ ID NO: 1) peptide sequence derived from MAGE-A4, when presented in the HLA-A*02-GVYDGREHTV antigen complex.

The CD8α co-receptor comprised in ADP-A2M4CD8 SPEAR™ T cells is designed to provide additional functionality to CD4 T cells. Because CD4+ T cells have a weak effector function in response to Class I antigens, a CD8α co-receptor was introduced alongside the TCR, in order to increase TCR binding avidity and enhance the polyfunctional response of engineered CD4+ T cells against MAGE-A4 positive tumour. The co-expression of CD8α adds CD8+ killer T cell capability to CD4+ helper T cells, while also maintaining/enhancing the helper cell capabilities of CD4+ T cells. The addition of a CD8α co-receptor directly impacts TCR binding to the HLA-peptide complex in CD4+ T cells, enhancing CD4+ T cell effector function. ADP-A2M4CD8 SPEAR™ T cells are therefore designed to improve upon ADP-A2M4 expressing T cells.

This has been confirmed in preclinical in vitro assays, in which ADP-A2M4CD8 showed a clear improvement in T cell activation (when cultured with antigen positive cells) relative to ADP-A2M4 expressing T cells, as measured by increased CD40L surface expression, particularly in the CD4+ fraction. When dendritic cells (DCs) were included in co-cultures, a marked improvement was seen with ADP-A2M4CD8 T cells. Cytokine release from both DCs (IL-12, MIG) and T cells (IFNy, IL-2 and other Th1) was improved compared to cultures containing the ADP-A2M4 cells. Additionally, a conversion of CD4+ T cells was seen, from being unable to kill MAGE-A4 positive 3D microspheres, to having an effective cytotoxic function when transduced with ADP-A2M4CD8. Therefore, CD4+ T cells transduced with ADP-A2M4CD8 display not only CD4+ helper functions, but also improved T cell effector functions.

Despite this improvement, preliminary results of one trial indicated that 55% of patients did not respond to ADP-A2M4CD8 therapy. A regime that improves the response to ADP-A2M4CD8 therapy is therefore desired. This Example concerns improvement of the response to ADP-A2M4CD8 therapy using an agent that helps to maintain an activated T cell response.

In this respect, a binding antagonist of the PD-L1/PD-1 axis is considered. PD-1 is expressed on T cells, and PD-L1 is expressed on a variety of different cancer cells. The physiologic role of PD-1 is to maintain T cell homeostasis by limiting T cell activation and proliferation. Ligation of PD-L1 to PD-1 transmits an inhibitory exhaustion signal that reduces cytokine production and inhibits T cell proliferation. In particular, when a T cell experiences coincident TCR and PD-1 binding, PD-1-generated signals prevent phosphorylation of key TCR signalling intermediates, which terminates early TCR signalling and reduces activation of T cells. T cell effector functions and transition to memory T cells are impaired. In this way, the adaptive anti-cancer immune response is limited and tumour resistance is promoted.

It is hypothesised that PD-1 pathway blockade may improve responses to ADP-A2M4CD8 therapy.

### Materials and Methods

Subjects were selected for investigation of the effects of PD-1 pathway blockade on responses to ADP-A2M4CD8 therapy. In brief, subjects having a histologically or cytogenetically confirmed diagnosis of urothelial cancer, oesophageal, oesophagogastric junction cancer, gastric cancer, non-small cell lung carcinoma (NSCLC), head and neck carcinoma, ovarian carcinoma, melanoma, or endometrial carcinoma were pre-screened to determine appropriate human leukocyte antigen (HLA) and tumour antigen status. For inclusion in the study, subjects must be positive for HLA-A*02:01, HLA-A*02:03, or HLA-A*02:06, or another HLA-A*02 allele having the same protein sequence in the peptide binding domains. Patients who were HLA-A*02:05 positive were excluded from the study, because alloreactivity from ADP-A2M4 and from ADP-A2M4CD8 was seen towards two HLA-A*02:05 positive cell lines. Patients with either HLA-A*02:07 or any A*02 null allele as the sole HLA-A*02 allele were also excluded due to decreased activity with these alleles. Subjects must also have a tumour that shows MAGE-A4 expression defined as ≥30% of tumour cells that are ≥2+ by immunohistochemistry (IHC).

Autologous cells were collected from enrolled subjects by leukapheresis for processing and manufacture into ADP-A2M4CD8. The heterologous TCR comprised in ADP-A2M4CD8 T cells comprises an alpha chain sequence as set out in SEQ ID NO: 2 and a beta chain sequence as set out in SEQ ID NO: 3. The heterologous CD8 co-receptor comprised in ADP-A2M4CD8 T cells comprises two CD8 alpha chains, each comprising an amino acid sequence of SEQ ID NO: 10. The surface-expressed heterologous TCR and surface-expressed heterologous CD8 co-receptor do not comprise the signal sequence.

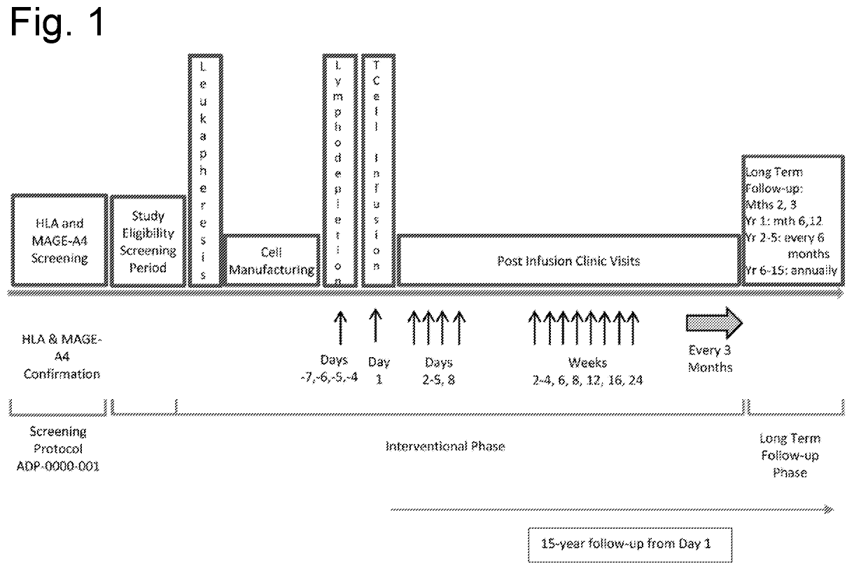

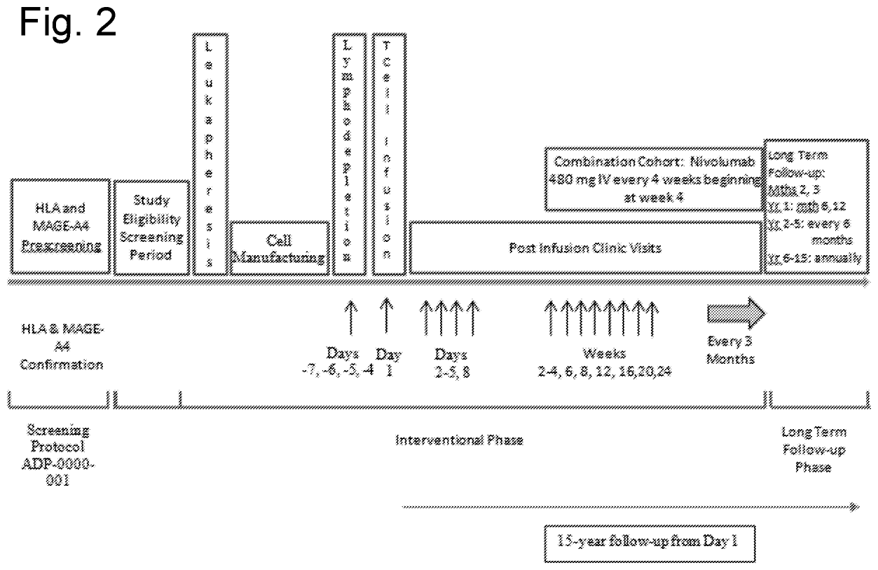

A baseline tumour assessment was obtained prior to treatment. Then, subjects were provided with lymphodepleting chemotherapy using fludarabine and cyclophosphamide (Day −7 through Day −4). Subjects received a single intravenous infusion of ADP-A2M4CD8 on Day 1. Approximately 60 subjects were treated in this way (Groups 1 to 3 in Table 1 below, and FIG. 1). An additional group of subjects, Group 4, were additionally treated with a PD-1 axis antagonist (nivolumab, and FIG. 2).

TABLE 1

| | | | Dose of | |
|---|---|---|---|---|
| | No. of | Transduced | cyclophosphamide | |
| Group | subjects | cells | and fludarabine | Interval for safety review |
| 1 | 3 to 6 | 1 × 10⁹ | Cy: 600 mg/m²/d | Minimum 14-day |
| | | (range: 0.8 × | Days −7, −6, and −5 | observation period after first |
| | | 10⁹ to | Flu: 30 mg/m²/d | subject prior to the start of |
| | | 1.2 × 10⁹) | Days −7, −6, −5 and −4 | lymphodepletion in the |
| | | | | second and third subjects |
| 2 | 3 to 6 | 5 × 10⁹ | Cy: 600 mg/m²/d | Minimum 14-day |
| | | (range: 1.2 × | Days −7, −6, and −5 | observation period after first |
| | | 10⁹ to | Flu: 30 mg/m²/d | subject prior to the start of |
| | | 6.0 × 10⁹) | Days −7, −6, −5 and −4 | lymphodepletion in the |
| | | | | second and third subjects |
| 3 | Up to 60 | | Cy: 600 mg/m²/d | No predetermined |
| | (including | | Days −7, −6, and −5 | observation period |
| | dose | | Flu: 30 mg/m²/d | |
| | escalation) | | Days −7, −6, −5 and −4 | |
| 4 | Up to 30 | 1 × 10⁹ | Cy: 600 mg/m²/d | Dosing of nivolumab in the |
| | | to | Days −7, −6, and −5 | first two subjects will be |
| | | 10 × 10⁹ | Flu: 30 mg/m²/d | staggered. Dosing of the |
| | | | Days −7, −6, −5 and −4 | second subject will be |
| | | | | delayed until the first subject |
| | | | | has been observed for at least |
| | | | | 14 days after nivolumab |
| | | | | administration. |

The initial dose selected for ADP-A2M4CD8 was 1×10⁹ transduced cells (Range: 0.8×10⁹-1.2×10⁹ transduced cells). To date, doses ranging from 0.1×10⁹ to 10×10⁹ ADP-A2M4 cells have been used in the clinic. Post infusion peak expansion of ADP-A2M4 cells was low and persistence was transient at doses below 1×10⁹, providing no potential for biological activity. Although the target antigen for both ADP-A2M4 and ADP-A2M4CD8 is MAGE-A4, the effect of the CD8α modification in humans was unknown. Therefore, to maintain a positive benefit: risk balance the starting dose in the study is selected to be potentially efficacious but below the tolerated doses with ADP-A2M4. The initial dose for ADP-A2M4CD8 is further supported by experience with of NY-ESO-1c259T in patients with synovial sarcoma. Subjects received a median-infused NY-ESO-1c259T cell dose of 3.6×10⁹ (range, 0.45×10⁹ to 14.4×10⁹) transduced cells [Araujo, 2019]. Two subjects who received a dose below 1×10⁹ did not show a response and had progressive disease (PD) by Week 12 post infusion, indicating that responses are more likely to be observed at doses at or above 1×10⁹ transduced T cells. Subjects were monitored immediately post-infusion monitoring (Day 1 through Day 8). Subjects are monitored weekly until Week 4 post-infusion. Then, subjects are monitored at Weeks 6, 8, 12, 16, and 24 and at least every 3 months thereafter until disease progression. Tumour response is assessed according to response evaluation criteria in solid tumours (RECIST) v1.1. Additional data collected included:
Core needle biopsies, to directly evaluate the “immune landscape” inside the tumour at baseline and during the course of the study.
Cytokine levels in the serum at baseline and during the course of the study.
Humoral immune responses to tumour antigens at baseline and during the course of the study, using serum.
Antibodies to ADP-A2M4CD8 at baseline and during the course of the study, using serum.
Soluble markers representing the tumour and its microenvironment, using liquid biopsies. For instance, markers of circulating tumour cells (CTCs), exosome, and cell-free DNA (cfDNA) produced by dying tumour cells) may be used to monitor both the molecular signature of the tumour burden (including the expression of the target antigen) and the immune response. The analysis of such soluble markers allows estimation and genetic profiling of the global tumour burden, including expression of MAGE-A4 mRNA and mutational profiling. The analysis of such soluble markers also allows systemic assessment of the immune response.
The phenotype and activity of the gene-modified T cells before and after infusion. The relevant assays may be performed using blood and, if resection is performed, tumour. The assays include: (i) phenotype analysis for determination of T-cell lineages in cell product and in the blood (and, if resection performed, tumour) post-infusion; (ii) quantitation of the senescence and activation status of immune subsets from PBMCs; (iii) analysis of gene expression or epigenetic profile to reflect phenotype and functional state of the cells; and/or (iv) direct functional assessment of the cells.
Persistence of infused engineered cells, and correlation with therapeutic effect. Persistence may be determined by the copies of gene-modified DNA per μg DNA, and/or data on the number of transduced cells per μL or relative to total lymphocyte number. Well-established methodologies include (i) quantitation of ADP-A2M4CD8 cells by quantitative PCR of transgene from DNA extracted from frozen PBMCs, and (ii) quantitation and phenotyping of ADP-A2M4CD8 cells by flow cytometry, DNA and RNA analysis from frozen PBMCs.

### Results

Across the study, doses of up to 10×10⁹ ADP-A2M4CD8 were administered and well tolerated. Treatment emergent adverse events (TEAEs) were consistent with the underlying diseases and known adverse event profile with non-myeloablative lymphodepletion or cancer immunotherapy. No dose-limiting toxicities were noted. Ten subjects reported serious adverse events that were deemed related to ADP-A2M4CD8. Most of the adverse events noted were consistent with those typically experienced by cancer subjects undergoing cytotoxic chemotherapy or cancer immunotherapy.

As of 24 May 2021, 20 subjects (1 with MRCLS, 5 with EGJ cancers, 6 with ovarian cancer, 2 with head and neck squamous cell carcinoma, and 2 with esophageal cancer, 1 with melanoma, 1 with NSCLC, 1 with synovial sarcoma and 1 with urothelial cancer) had received ADP-A2M4CD8 (range 1.0 to 5.9 billion transduced cells), with treatment benefits/tumour regression observed in patients with EGJ cancer, H&N cancer, oesophageal cancer and ovarian cancer. At this date, overall, across all dose groups and tumour types, the best overall response (BOR) was 1 subject (5%) with a confirmed complete response, 4 subjects (20%) with a confirmed partial response (cPR), 2 subjects (10%) with an unconfirmed partial response (uPR), 8 subjects (40%) with SD, 3 (15%) subjects with PD, and 1 (5%) subject as missing this assessment. One subject with EGJ cancer had a cPR per RECIST and had progression free survival (PFS) >36 weeks as of the 24 May 2021 cut-off date. Two head and neck cancer patients had a cPR. Tumour regression (sum of diameters of target lesions with a nadir) of 16.3% reduction from baseline was observed in the ovarian cancer patient.

Studies of SPEAR™ T cells show that PD-1 is expressed on CD8+ and CD4+ T cells in the majority of apheresis products, and in both transduced and non-transduced T cells in the manufactured product. In addition, in one subject with synovial sarcoma and another with ovarian cancer, induction of intra-tumoural PD-L1 was observed early after T cell infusion and was associated with an increase in transduced and non-transduced SPEAR™ T cells. Furthermore, in vitro experiments have shown that in multiple donors, PD-1 blockade with pembrolizumab partially restores IFN-gamma production by pre-activated A2M4 SPEAR™ T cells during re-stimulation. PD-1/PD-L1 blockade has also been demonstrated enhance the effect of CAR-T therapy in vivo and ex vivo in hematologic malignancy and solid tumours.

PD-1 pathway blockade may therefore maintain the function of infused ADP-A2M4CD8 T cells, such as effector function and ability to transition to memory T cells. The generation of memory T cells is advantageous in the treatment of tumours, because their presence of in a subject may provide an ongoing anti-tumour dynamic that keeps tumours in check in the long term (e.g. months to years), even in the absence of continued ADP-A2M4CD8 T cell therapy. Furthermore, the present inventors propose that PD-1 pathway blockade may improve patient responses to ADP-A2M4CD8 T cell therapy by engaging the patient's own immune system. In particular, a PD-1 axis antagonist may engage endogenous T cells, such as those infiltrating the tumour microenvironment. PD-1 pathway blockade may maintain the function of these endogenous T cells, such as effector function and ability to transition to memory T cells. Accordingly, PD-1 pathway blockade may (i) sustain the function of ADP-A2M4CD8 T cells, and/or (ii) maintain the function of endogenous T cells such that they may contribute to the anti-tumour response. In these ways, PD-1 pathway blockade may enhance and/or prolong the effect of a single dose of ADP-A2M4CD8 T cells administered to a subject.

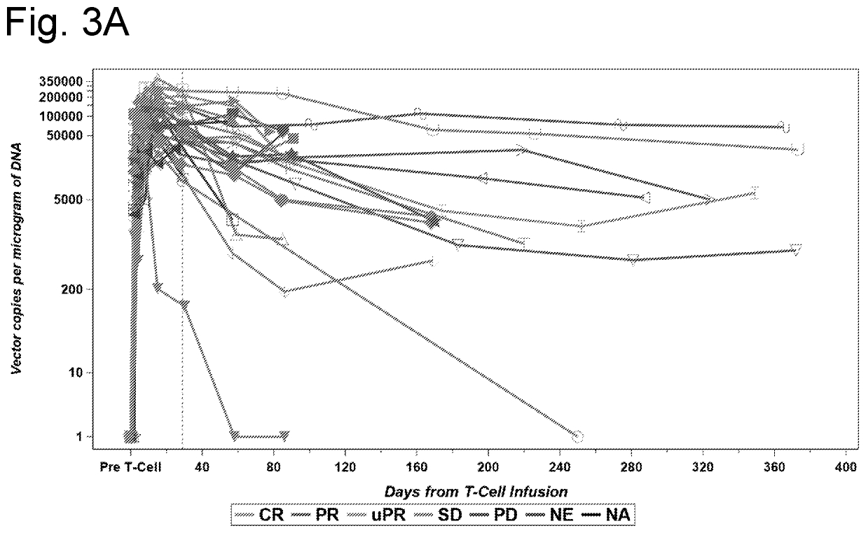

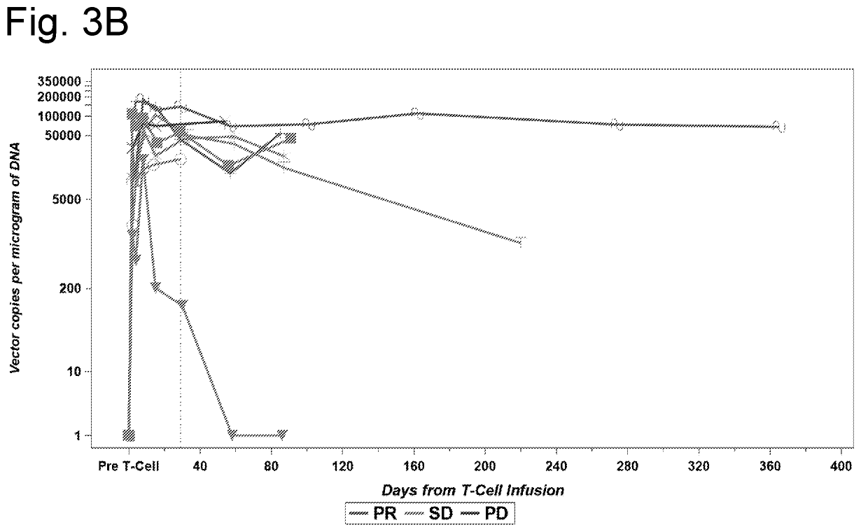

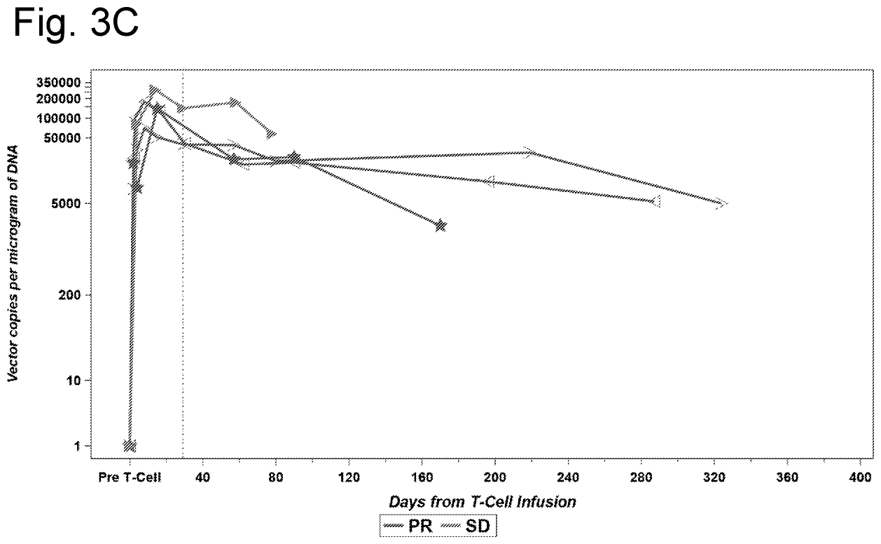

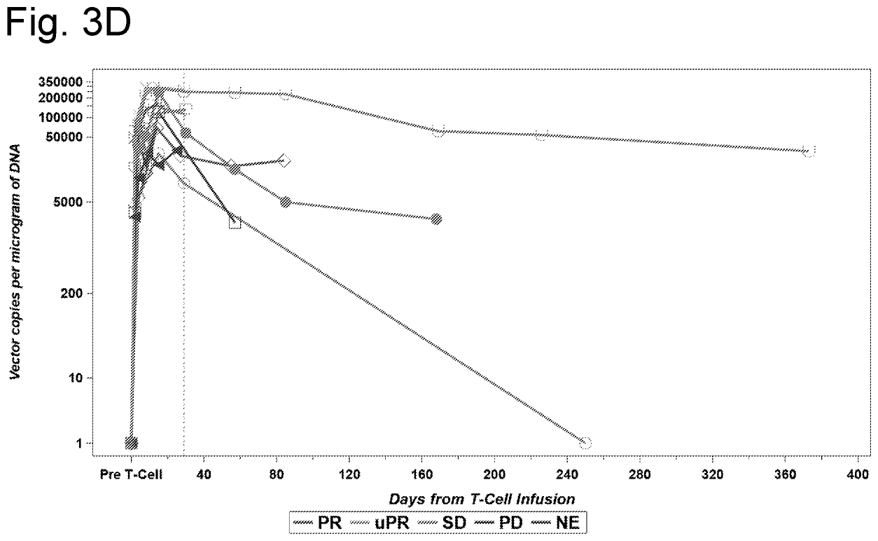

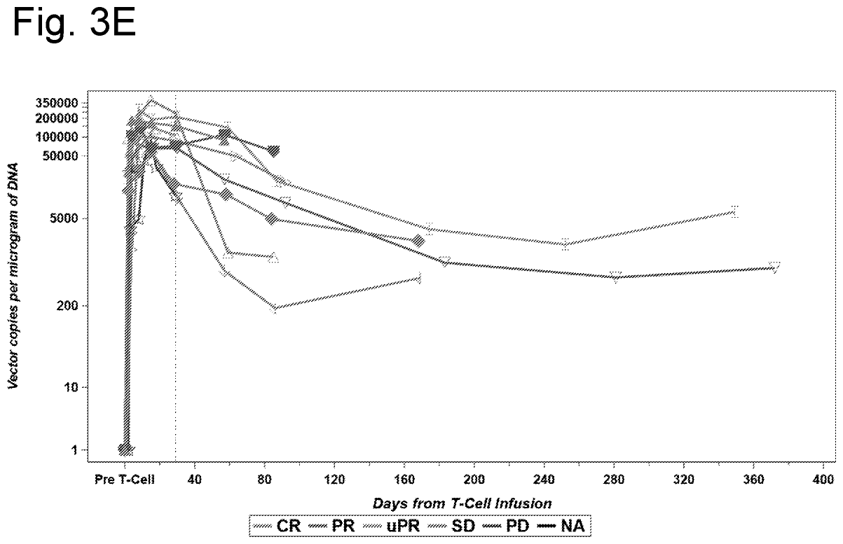

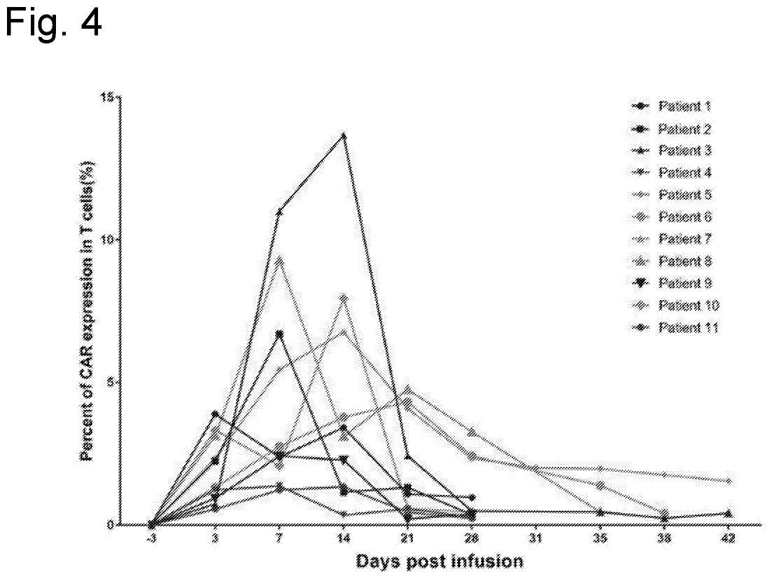

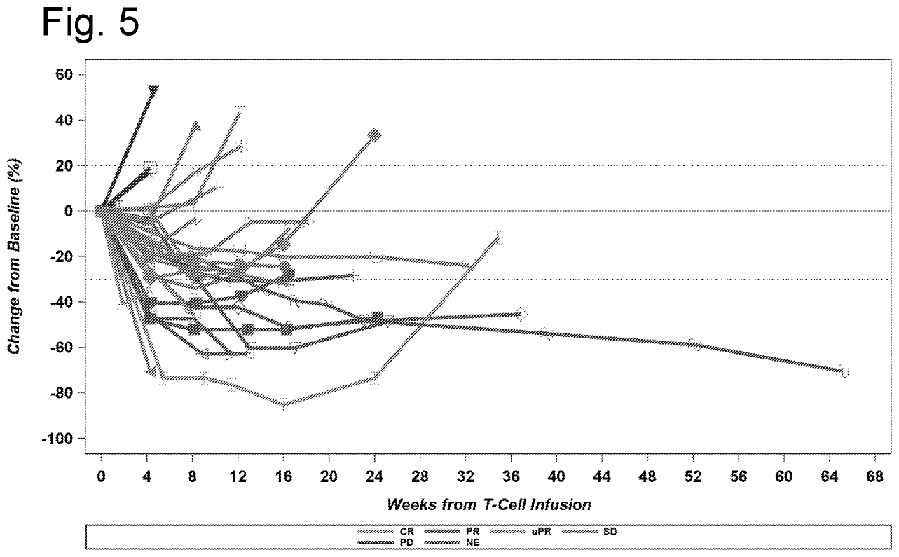

In this respect, the present study has enabled an optimal dosage regime to be identified for administering a PD-1 axis inhibitor in combination with ADP-A2M4CD8 T cells. Table 2 below and FIG. 3 summarise the peak persistence of ADP-A2M4CD8 T cells, and time to peak persistence, in subjects who received an infusion of ADP-A2M4CD8 T cells. FIG. 4, taken from the prior art, provides comparative data relating to persistence of a single infusion of CAR T cells. FIG. 5 shows the effect of a single infusion of ADP-A2M4CD8 T cells on target lesions (tumours).

TABLE 2

| | | | | Expansion | |
|---|---|---|---|---|---|
| | | Group 1 | Group 2 | Group | |
| | | (N = 3; 0.8 × 10⁹ | (N = 3; 1.2 × 10⁹ | (N = 30; 1.2 × 10⁹ | |
| | | to 1.2 × 10⁹ T | to 6.0 × 10⁹ T | to 10.0 × 10⁹ T | Overall |
| | Statistics | cells) | cells) | cells) | (N = 36) |
| Peak | n | 3 | 3 | 27 | 33 |
| Persistence | Mean | 53048.23 | 85693.27 | 149255.63 | 134731.11 |
| (copies/ | Median | 62113.00 | 70946.90 | 138100.10 | 120784.80 |
| microgram | Standard | 22090.124 | 41319.707 | 93782.251 | 91171.170 |
| DNA) | Deviation | | | | |
| | Min, Max | 27867.7, 69164.0 | 53769.98, 132363.1 | 20739.2, 387570.5 | 20739.2, 387570.5 |
| | Missing | 0 | 0 | 3 | 3 |
| Time to | n | 3 | 3 | 27 | 33 |
| Peak | Mean | 14.67 | 15.67 | 13.74 | 14.00 |
| Persistence | Median | 15.00 | 9.00 | 15.00 | 15.00 |
| (Days) | Standard | 0.577 | 12.423 | 10.189 | 9.715 |
| | Deviation | | | | |
| | Min, Max | 14.0, 15.0 | 8.0, 30.0 | 2.0, 54.0 | 2.0, 54.0 |
| | Missing | 0 | 0 | 3 | 3 |

The data show that ADP-A2M4CD8 T cells optimally persist for a long period of more than 24 weeks (see Table 2 and FIG. 3). This is in contrast to CAR T cells, which are demonstrated in the prior art to persist only at low levels after an initial peak 7-14 days post-infusion (FIG. 4).

The data further demonstrate that tumours show an early response to ADP-A2M4CD8 T cells. The first tumour assessment was performed at week 4. FIG. 5 shows that, even at this early time point, ADP-A2M4CD8 T cells induced anti-tumour activity that resulted in a reduction in tumour burden, in some cases meeting RECIST criteria for partial response.

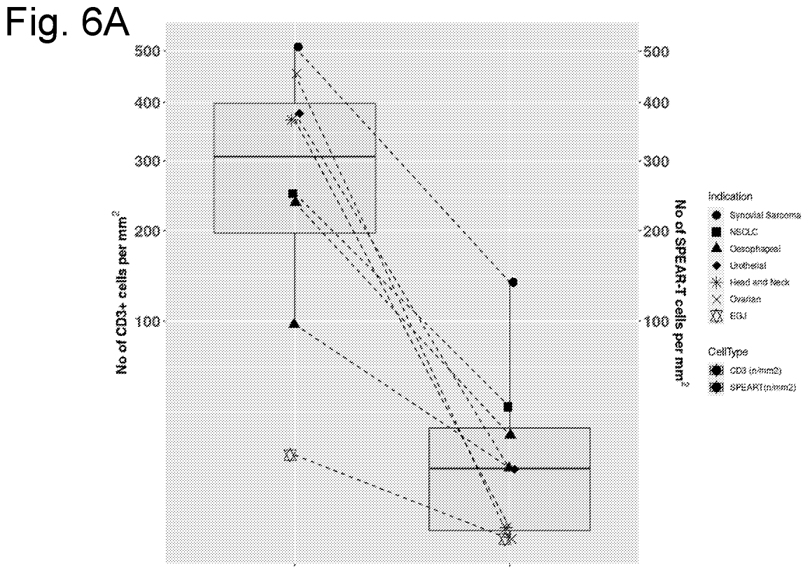

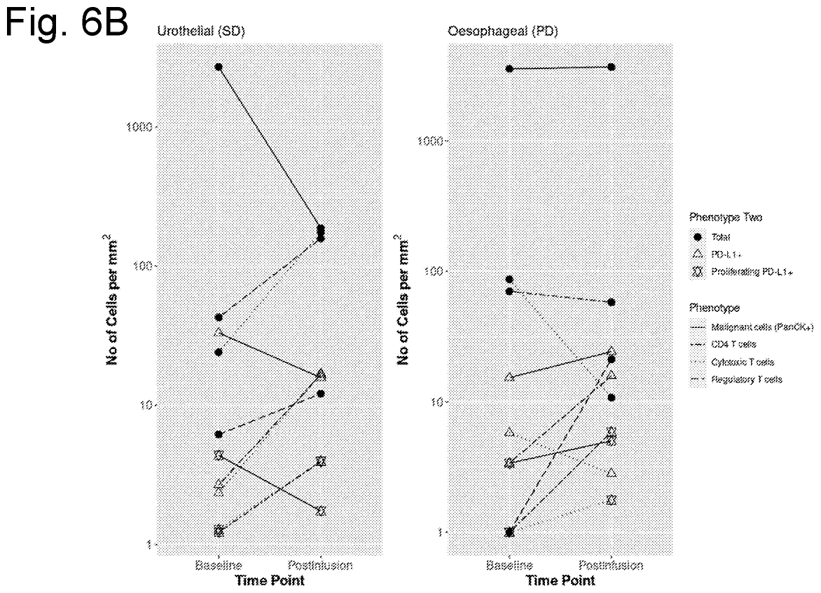

The inventors have also found that intra-tumoural ADP-A2M4CD8 T cells are detected in 75% of evaluable biopsies obtained from patients administered with ADP-A2M4CD8 T cells. FIG. 6A demonstrates that tumours contain other T cells, in addition to the A2M4CD8 T cells. That is, tumours contain endogenous T cells from the patient, as well as the administered therapeutic T cells. As shown in FIG. 6B, administration of ADP-A2M4CD8 T cells may increase infiltration of the patient's own T cells to the tumour. In particular, the boxplots in FIG. 6B show the changes in number of malignant cells, CD4 helper T cells, cytotoxic T cells and regulatory T cells from baseline to post-ADP-A2M4CD8 T cell infusion. Phenotype is also considered. In a patient with urothelial cancer having stable disease (SD) post-infusion, the native (PDL1− and PDL1−Ki67−) forms of malignant cells decrease from baseline to post-infusion. PDL1+ and proliferating PDL1+ malignant cells decreased more slowly from baseline to post-infusion, consistent with their stronger immune-suppressive phenotype and associated reduced susceptibility to immune cell killing. Native forms of CD4 T helper cells, cytotoxic T cells regulatory T cells increased post-infusion, and PDL1+ CD4 T cells and PDL1+ cytotoxic T cells showed slightly higher rate of increase. In a patient with oesophageal cancer having progressive disease (PD), the native (PDL1− and PDL1−Ki67−) forms of malignant cells very slightly increased from baseline to post-infusion. PDL1+ and proliferating PDL1+ malignant cells showed an increased rate of density change compared to native malignant cells, consistent with their stronger immune-suppressive phenotype and associated reduced susceptibility to immune cell killing. Native forms of CD4 T helper cells and especially cytotoxic T cells decreased post-baseline, as expected for progressive disease. Regulatory T cells showed a steep increase from baseline to post-infusion. PDL1+ cytotoxic T cells showed slightly slower rate of decrease from baseline to post-infusion, and PDL1+ proliferating cytotoxic T cells even showed an increase in density. PDL1+ and proliferating PDL1+ CD4 T cells showed an increase in density, from baseline to post-infusion compared to native forms. The data presented in Table 2 and FIGS. 3 to 6, and described above, demonstrate that modified and endogenous TILs exist by week four following administration of ADP-A2M4CD8 T cells, and that there is concurrent reduction in tumour size. Thus, the data indicate that a PD-PD-L1-PD-L2 axis inhibitor may optimally be administered at or around four weeks after infusion of ADP-A2M4CD8 T cells. In this respect, many patients in early clinical studies using either anti-PD1 or anti-PD-L1 antibody have demonstrated objective clinical responses. The mechanism for such response is considered to be sustained T cell activity/functionality of both engineered and natural-occurring T cells, by delaying T cell exhaustion mediated by PD-PD-L1-PD-L2 axis signalling. Thus, administration of a PD-PD-L1-PD-L2 axis inhibitor at or around week four is considered to prolong effector function and ultimately increase the durability of activated T cells.

Treatment with a PD-PD-L1-PD-L2 axis inhibitor at or around the four week point also has safety and tolerability positive implications. Emerging data shows that after four weeks most patients are recovered from any safety event related to either lymphodepletion (cytopenia) or ADP-A2M4CD8 T cells infusion (CRS). Thus, administration of a PD-PD-L1-PD-L2 axis inhibitor at or around week 4 is also considered to prevent overlapping toxicities and to be well tolerated by the patients. It is surprising that a PD-PD-L1-PD-L2 axis inhibitor may effectively be administered at or around week four after administration of ADP-A2M4CD8 T cells. Prior art concerning the administration of T cells with a PD-PD-L1-PD-L2 axis inhibitor (e.g. Cao et al. (2019), Anti-CD19 Chimeric Antigen Receptor T Cells in Combination With Nivolumab Are Safe and Effective Against Relapsed/Refractory B-Cell Non-hodgkin Lymphoma, Frontiers in Oncology, 9:767, doi: 10.3389/fonc.2019.00767) indicates that the administered T cells peak at around 7-14 days and are subsequently reduced to very low levels. The prior art therefore teaches that a PD-PD-L1-PD-L2 axis inhibitor should be administered shortly after therapeutic T cells, such as at day 3.

SEQUENCE LISTING

SEQ ID NO: 1-MAGE-A4230-239
GVYDGREHTV
SEQ ID NO: 2-MAGE-A4 TCR alpha chain (CDRs bold underlined, signal
sequence italic underlined)
MKKHLTTFLVILWLYFYRGNGKNQVEQSPQSLIILEGKNCTLQCNYTVSPFSNLRWYKQDT
GRGPVSLTILTFSENTKSNGRYTATLDADTKQSSLHITASQLSDSASYICVVSGGTDSWGK
LQFGAGTQVVVTPDIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETDTN
LNFQNLSVIGFRILLLKVAGFNLLMTLRLWSSGSRAKR
SEQ ID NO: 3-MAGE-A4 TCR beta chain (CDRs bold underlined, signal sequence
italic underlined)
MASLLFFCGAFYLLGTGSMDADVTQTPRNRITKTGKRIMLECSQTKGHDRMYWYRQDPGLG
LRLIYYSFDVKDINKGEISDGYSVSRQAQAKFSLSLESAIPNQTALYFCATSGQGAYEEQF
FGPGTRLTVLEDLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMAMVK
RKDSRG
SEQ ID NO: 4-MAGE-A4 TCR alpha chain CDR1
VSPFSN
SEQ ID NO: 5-MAGE-A4 TCR alpha chain CDR2
LTFSEN
SEQ ID NO: 6-MAGE-A4 TCR alpha chain CDR3
CVVSGGTDSWGKLQF
SEQ ID NO: 7-MAGE-A4 TCR beta chain CDR1
KGHDR
SEQ ID NO: 8-MAGE-A4 TCR beta chain CDR2
SFDVKD
SEQ ID NO: 9-MAGE-A4 TCR beta chain CDR3
CATSGQGAYEEQFF
SEQ ID NO: 10-CD8 alpha chain (CDRs bold underlined, signal sequence italic
underlined)
MALPVTALLLPLALLLHAARPSQFRVSPLDRTWNLGETVELKCQVLLSNPTSGCSWLFQPR
GAAASPTFLLYLSQNKPKAAEGLDTQRFSGKRLGDTFVLTLSDFRRENEGYYFCSALSNSI
MYFSHFVPVFLPAKPTTTPAPRPPTPAPTIASQPLSLRPEACRPAAGGAVHTRGLDFACDI
YIWAPLAGTCGVLLLSLVITLYCNHRNRRRVCKCPRPVVKSGDKPSLSARYV
SEQ ID NO: 11-CD8 alpha chain CDR1
VLLSNPTSG
SEQ ID NO: 12-CD8 alpha chain CDR2
YLSQNKPK
SEQ ID NO: 13-CD8 alpha chain CDR3
LSNSIM
SEQ ID NO: 14-PD1-Human Programmed cell death protein (Homo sapiens)
MQIPQAPWPVVWAVLQLGWRPGWFLDSPDRPWNPPTFSPALLVVTEGDNATFTCSFSNTS
ESFVLNWYRMSPSNQTDKLAAFPEDRSQPGQDCRFRVTQLPNGRDFHMSVVRARRNDSGT
YLCGAISLAPKAQIKESLRAELRVTERRAEVPTAHPSPSPRPAGQFQTLVVGVVGGLLGS
LVLLVWVLAVICSRAARGTIGARRTGQPLKEDPSAVPVFSVDYGELDFQWREKTPEPPVP
CVPEQTEYATIVFPSGMGTSSPARRGSADGPRSAQPLRPEDGHCSWPL
SEQ ID NO: 15-PD1L1-Human Programmed cell death 1 ligand 1 (Homo sapiens)
MRIFAVFIFMTYWHLLNAFTVTVPKDLYVVEYGSNMTIECKFPVEKQLDLAALIVYWEME
DKNIIQFVHGEEDLKVQHSSYRQRARLLKDQLSLGNAALQITDVKLQDAGVYRCMISYGG
ADYKRITVKVNAPYNKINQRILVVDPVTSEHELTCQAEGYPKAEVIWTSSDHQVLSGKTT
TTNSKREEKLFNVTSTLRINTTTNEIFYCTFRRLDPEENHTAELVIPELPLAHPPNERTH
LVILGAILLCLGVALTFIFRLRKGRMMDVKKCGIQDTNSKKQSDTHLEET
SEQ ID NO: 16-PD1L2-Human Programmed cell death 1 ligand 2 (Homo sapiens)
MIFLLLMLSLELQLHQIAALFTVTVPKELYIIEHGSNVTLECNFDTGSHVNLGAITASLQ
KVENDTSPHRERATLLEEQLPLGKASFHIPQVQVRDEGQYQCIIIYGVAWDYKYLTLKVK
ASYRKINTHILKVPETDEVELTCQATGYPLAEVSWPNVSVPANTSHSRTPEGLYQVTSVL
RLKPPPGRNFSCVFWNTHVRELTLASIDLQSQMEPRTHPTWLLHIFIPFCIIAFIFIATV
IALRKQLCQKLYSSKDTTKRPVTTTKREVNSAI
SEQ ID NO: 17-Nivolumab Heavy Chain Sequence
QVQLVESGGGVVQPGRSLRLDCKASGITFSNSGMHWVRQAPGKGLEWVAVIWYDGSKRYY
ADSVKGRFTISRDNSKNTLFLQMNSLRAEDTAVYYCATNDDYWGQGTLVTVSSASTKGPS
VFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTC
LVKGFYPSDIAVEWESNGQPENNYKTTPPVLDSDGSFFLYSRLTVDKSRWQEGNVFSCSV
MHEALHNHYTQKSLSLSLGK
SEQ ID NO: 18-Nivolumab Light Chain Sequence
EIVLTQSPATLSLSPGERATLSCRASQSVSSYLAWYQQKPGQAPRLLIYDASNRATGIPA
RFSGSGSGTDFTLTISSLEPEDFAVYYCQQSSNWPRTFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 19-Pembrolizumab Heavy Chain Sequence
QVQLVQSGVEVKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYWGQGTTVTVSS
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
SEQ ID NO: 20-Pembrolizumab Light Chain Sequence
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 21-Cemiplimab Heavy Chain Sequence
EVQLLESGGVLVQPGGSLRLSCAASGFTFSNFGMTWVRQAPGKGLEWVSGISGGGRDTYF
ADSVKGRFTISRDNSKNTLYLQMNSLKGEDTAVYYCVKWGNIYFDYWGQGTLVTVSSAST
KGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFLGGPSVFLF
PPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVDKSRWQEGNVF
SCSVMHEALHNHYTQKSLSLSLGK
SEQ ID NO: 22-Cemiplimab Light Chain Sequence
DIQMTQSPSSLSASVGDSITITCRASLSINTFLNWYQQKPGKAPNLLIYAASSLHGGVPS
RFSGSGSGTDFTLTIRTLQPEDFATYYCQQSSNTPFTFGPGTVVDFRRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 23-Durvalumab Heavy Chain Sequence
EVQLVESGGGLVQPGGSLRLSCAASGFTFSRYWMSWVRQAPGKGLEWVANIKQDGSEKYY
VDSVKGRFTISRDNAKNSLYLQMNSLRAEDTAVYYCAREGGWFGELAFDYWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPEFEG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREPQVYTLPPSRE
EMTKNQVSLTCLVKGFYPSDIAVEWESNGQPENNYKTTPPVLDSDGSFFLYSKLTVDKSR
WQQGNVFSCSVMHEALHNHYTQKSLSLSPGK
SEQ ID NO: 24-Durvalumab Light Chain Sequence
EIVLTQSPGTLSLSPGERATLSCRASQRVSSSYLAWYQQKPGQAPRLLIYDASSRATGIP
DRFSGSGSGTDFTLTISRLEPEDFAVYYCQQYGSLPWTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 25-Atezolizumab Heavy Chain Sequence
EVQLVESGGGLVQPGGSLRLSCAASGFTFSDSWIHWVRQAPGKGLEWVAWISPYGGSTYY
ADSVKGRFTISADTSKNTAYLQMNSLRAEDTAVYYCARRHWPGGFDYWGQGTLVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 26-Atezolizumab Light Chain Sequence
DIQMTQSPSSLSASVGDRVTITCRASQDVSTAVAWYQQKPGKAPKLLIYSASFLYSGVPS
RFSGSGSGTDFTLTISSLQPEDFATYYCQQYLYHPATFGQGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 27-Avelumab Heavy Chain Sequence
EVQLLESGGGLVQPGGSLRLSCAASGFTFSSYIMMWVRQAPGKGLEWVSSIYPSGGITFY
ADTVKGRFTISRDNSKNTLYLQMNSLRAEDTAVYYCARIKLGTVTTVDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 28-Avelumab Light Chain Sequence
QSALTQPASVSGSPGQSITISCTGTSSDVGGYNYVSWYQQHPGKAPKLMIYDVSNRPSGV
SNRFSGSKSGNTASLTISGLQAEDEADYYCSSYTSSSTRVFGTGTKVTVLGQPKANPTVT
LFPPSSEELQANKATLVCLISDFYPGAVTVAWKADGSPVKAGVETTKPSKQSNNKYAASS
YLSLTPEQWKSHRSYSCQVTHEGSTVEKTVAPTECS
SEQ ID NO: 29-BMS 936559 (MDX-1105) Heavy Chain Sequence
QVQLVQSGAEVKKPGSSVKVSCKTSGDTFSTYAISWVRQAPGQGLEWMGGIIPIFGKAHYAQKFQG
RVTITADESTSTAYMELSSLRSEDTAVYFCARKFHFVSGSPFGMDVWGQGTTVTVSSASTKGPSVF
PLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKT
SEQ ID NO: 30-BMS 936559 (MDX-1105) Light Chain Sequence
EIVLTQSPATLSLSPGERATLSCRASQSVSSYLAWYQQKPGQAPRLLIYDASNRATGIPARFSGSG
SGTDFTLTISSLEPEDFAVYYCQQRSNWPTFGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQ
GLSSPVTKSFNRGEC
SEQ ID NO: 31-Dostarlimab Heavy Chain Sequence
EVQLLESGGGLVQPGGSLRLSCAASGFTFSSYDMSWVRQAPGKGLEWVSTISGGGSYTYYQDSVKG
RFTISRDNSKNTLYLQMNSLRAEDTAVYYCASPYYAMDYWGQGTTVTVSSASTKGPSVFPLAPCSR
STSESTAALGCLVKDYFPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 32-Dostarlimab Light Chain Sequence
DIQLTQSPSFLSAYVGDRVTITCKASQDVGTAVAWYQQKPGKAPKLLIYWASTLHTGVPSRFSGSG
SGTEFTLTISSLQPEDFATYYCQHYSSYPWTFGQGTKLEIKRTVAAPSVFIFPPSDEQLKSGTASV
VCLLNNFYPREAKVQWKVDNALQSGNSQESVTEQDSKDSTYSLSSTLTLSKADYEKHKVYACEVTH
QGLSSPVTKSFNRGEC

1. A method of treating cancer in an individual, comprising:
(a) administering to the individual a population of modified T cells comprising a heterologous CD8 co-receptor and a heterologous TCR capable of binding to MAGE-A4; and
(b) sustaining the function of (i) the population of modified T cells and/or (ii) endogenous T cells in the individual by administering an initial dose of a PD-1 axis binding antagonist to the individual about three weeks to about five weeks after step (a).

2. A population of modified T cells for use in a method of treating cancer in an individual, wherein the modified T cells comprise a heterologous CD8 co-receptor and a heterologous TCR capable of binding to MAGE-A4, and the method comprises:
(a) administering the population to the individual; and
(b) sustaining the function of (i) the population of modified T cells and/or (ii) endogenous T cells in the individual by administering an initial dose of a PD-1 axis binding antagonist to the individual about three weeks to about five weeks after step (a).

3. A PD-1 axis binding antagonist for use in a method of treating cancer in an individual, wherein the method comprises:
(a) administering to the individual a population of modified T cells comprising a heterologous CD8 co-receptor and a heterologous TCR capable of binding to MAGE-A4; and
(b) sustaining the function of (i) the population of modified T cells and/or (ii) endogenous T cells in the individual by administering an initial dose of the PD-1 axis binding antagonist to the individual about three weeks to about five weeks after step (a).

4. The method of claim 1, population for use of claim 2, or PD-1 axis binding antagonist for use of claim 3, wherein the initial dose of the PD-1 axis binding antagonist is administered to the individual about four weeks after administration of the population of modified T cells.

5. The method of claim 1 or 4, population for use of claim 2 or 4, or PD-1 axis binding antagonist for use of claim 3 or 4, wherein the method further comprises administering one or more further doses of the PD-1 axis binding antagonist, optionally wherein the one or more further doses are administered once every four weeks (Q4W) beginning four weeks from administration of the initial dose.

6. The method of any one of claims 1, 4 and 5, the population for use of any one of claims 2, 4 and 5, or the PD-1 axis binding antagonist for use of any one of claims 3 to 5, wherein administration of the PD-1 axis binding antagonist reduces exhaustion (i) within the population of modified T cells and/or within T cells descended from the population of modified T cells, and/or (ii) within endogenous T cells in the individual.

7. The method of any one of claims 1 and 4 to 6, the population for use of any one of claims 2 and 4 to 6, or the PD-1 axis binding antagonist for use of any one of claims 3 to 6, wherein the heterologous TCR binds to SEQ ID NO: 1.

8. The method of any one of claims 1 and 4 to 7, the population for use of any one of claims 2 and 4 to 7, or the PD-1 axis binding antagonist for use of any one of claims 3 to 7, wherein the heterologous TCR comprises an alpha chain amino acid sequence having at least 80% sequence identity to SEQ ID NO: 2.

9. The method of any one of claims 1 and 4 to 8, the population for use of any one of claims 2 and 4 to 8, or the PD-1 axis binding antagonist for use of any one of claims 3 to 8, wherein the heterologous TCR comprises a beta chain amino acid sequence having at least 80% sequence identity to SEQ ID NO: 3.

10. The method of any one of claims 1 and 4 to 9, the population for use of any one of claims 2 and 4 to 9, or the PD-1 axis binding antagonist for use of any one of claims 3 to 9, wherein the CD8 co-receptor is CD8α.

11. The method of any one of claims 1 and 4 to 10, the population for use of any one of claims 2 and 4 to 10, or the PD-1 axis binding antagonist for use of any one of claims 3 to 10, wherein the CD8 co-receptor comprises an amino acid sequence having at least 80% sequence identity to SEQ ID NO: 10.

12. The method of any one of claims 1 and 4 to 11, the population for use of any one of claims 2 and 4 to 11, or the PD-1 axis binding antagonist for use of any one of claims 3 to 11, wherein the modified T cells are autologous with respect to the individual.

13. The method, population for use, or PD-1 axis binding antagonist for use of claim 12, wherein the method comprises producing the population by:
(i) obtaining peripheral blood mononuclear cells (PBMCs) from the individual;
(ii) selecting T cells from the PBMCs; and
(iii) modifying the selected T cells to express a heterologous CD8 co-receptor and a heterologous TCR capable of binding to MAGE-A4.

14. The method of any one of claims 1 and 4 to 13, the population for use of any one of claims 2 and 4 to 13, or the PD-1 axis binding antagonist for use of any one of claims 3 to 13, wherein the method comprises administering lymphodepleting chemotherapy to the individual prior to administration of the population of modified T cells.

15. The method of any one of claims 1 and 4 to 14, the population for use of any one of claims 2 and 4 to 14, or the PD-1 axis binding antagonist for use of any one of claims 3 to 14, wherein the population comprises 0.8×10⁹ to 10×10⁹ modified T cells, optionally wherein the population comprises 0.8×10⁹ to 1.2×10⁹ modified T cells, 1.2×10⁹ to 6×10⁹ modified T cells, or 1.0×10⁹ to 10×10⁹ modified T cells.

16. The method of any one of claims 1 and 4 to 15, the population for use of any one of claims 2 and 4 to 15, or the PD-1 axis binding antagonist for use of any one of claims 3 to 15, wherein the population comprises about 1.0×10⁹ modified T cells, about 5.0×10⁹ modified T cells, or about 10×10⁹ modified T cells.

17. The method of any one of claims 1 and 4 to 16, the population for use of any one of claims 2 and 4 to 16, or the PD-1 axis binding antagonist for use of any one of claims 3 to 16, wherein the population of modified T cells is administered as a single dose.

18. The method of any one of claims 1 and 4 to 17, the population for use of any one of claims 2 and 4 to 17, or the PD-1 axis binding antagonist for use of any one of claims 3 to 17, wherein the population of modified T cells is administered intravenously.

19. The method of any one of claims 1 and 4 to 18, the population for use of any one of claims 2 and 4 to 18, or the PD-1 axis binding antagonist for use of any one of claims 3 to 18, wherein the PD-1 axis binding antagonist is a PD1 binding antagonist.

20. The method, population for use, or PD-1 axis binding antagonist for use of claim 19, wherein the PD1 binding antagonist is an antibody that binds PD-1.

21. The method, population for use, or PD-1 axis binding antagonist for use of claim 20, wherein the antibody is nivolumab.

22. The method of any one of claims 1 and 4 to 21, the population for use of any one of claims 2 and 4 to 21, or the PD-1 axis binding antagonist for use of any one of claims 3 to 21, wherein each dose of the PD-1 axis binding antagonist comprises 200 mg to 700 mg of the PD-1 axis binding antagonist.

23. The method, population for use, or PD-1 axis binding antagonist for use of claim 22, wherein each dose of the PD-1 axis binding antagonist comprises 300 mg to 600 mg, 400 mg to 500 mg, 450 mg to 500 mg, or 480 mg of the PD-1 axis binding antagonist.

24. The method of any one of claims 1 and 4 to 23, the population for use of any one of claims 2 and 4 to 23, or the PD-1 axis binding antagonist for use of any one of claims 3 to 23, wherein each dose of the PD-1 axis binding antagonist comprises 480 mg of nivolumab.

25. The method of any one of claims 1 and 4 to 24, the population for use of any one of claims 2 and 4 to 24, or the PD-1 axis binding antagonist for use of any one of claims 3 to 24, wherein the cancer expresses MAGE-A4 and/or is a solid tumour, optionally wherein the solid tumour is urothelial cancer, head and neck cancer, non-small cell lung cancer (NSCLC), oesophageal caner, oesophogastric cancer, gastric cancer, ovarian cancer, melanoma, or endometrial cancer.


SEQUENCE LISTING (32 sequences)

SEQ ID NO: 1 - protein, 10 aa, Homo sapiens
GVYDGREHTV

SEQ ID NO: 2 - protein, 282 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 3 - protein, 311 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - protein, 6 aa, synthetic construct
VSPFSN

SEQ ID NO: 5 - protein, 6 aa, synthetic construct
LTFSEN

SEQ ID NO: 6 - protein, 15 aa, synthetic construct
CVVSGGTDSWGKLQF

SEQ ID NO: 7 - protein, 5 aa, synthetic construct
KGHDR

SEQ ID NO: 8 - protein, 6 aa, synthetic construct
SFDVKD

SEQ ID NO: 9 - protein, 14 aa, synthetic construct
CATSGQGAYEEQFF

SEQ ID NO: 10 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 11 - protein, 8 aa, synthetic construct
LLSNPTSG

SEQ ID NO: 12 - protein, 8 aa, synthetic construct
YLSQNKPK

SEQ ID NO: 13 - protein, 6 aa, synthetic construct
LSNSIM

SEQ ID NO: 14 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 16 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 17 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 29 - protein, 229 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 30 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC